(19) United States
(12) Patent Application Publication
(10) Pub. No.: US 2024/0197589 A1
(43) Pub. Date: Jun. 20, 2024

(54) ORAL CARE COMPOSITIONS COMPRISING GERANYLGERANIOL AND METHODS OF USE THEREOF

(71) Applicant: AMERICAN RIVER NUTRITION, LLC, Hadley, MA (US)

(72) Inventors: Barrie TAN, Amherst, MA (US); Anne TRIAS, Amherst, MA (US); Jonathan Paul LIZOTTE, Palm Coast, FL (US)

(73) Assignee: AMERICAN RIVER NUTRITION, LLC, Hadley, MA (US)

(21) Appl. No.: 18/473,740

(22) Filed: Sep. 25, 2023

Related U.S. Application Data
(60) Provisional application No. 63/376,807, filed on Sep. 23, 2022.

(51) Int. Cl. A61K 8/34; A61K 8/19; A61K 8/24; A61K 8/35; A61K 8/37; A61K 8/60; A61K 8/66; A61K 8/73; A61K 8/92; A61K 8/9789; A61K 8/99; A61L 24/00; A61Q 11/00

(52) U.S. Cl. CPC A61K 8/342; A61K 8/19; A61K 8/24; A61K 8/345; A61K 8/355; A61K 8/37; A61K 8/604; A61K 8/66; A61K 8/731; A61K 8/922; A61K 8/9789; A61K 8/99; A61L 24/0015; A61Q 11/00; C12Y 304/22032; C12Y 304/22033; A61K 2800/30; A61L 2300/216

(57) ABSTRACT

The present embodiments relate to oral compositions comprising geranylgeraniol and to methods of using and of making said compositions. The oral compositions can be used in, for example, an oral rinse, a toothpaste, mouth spray, chewing gum, or impregnated in a disposable toothbrush. The compositions can be applied to the gum, alveolar jaw bone, tooth, tooth tissue, enamel, oral cavity, or tooth socket for many uses including, but not limited to, promoting gum growth and gum support, wound healing, and oral hygiene. Additionally, the oral compositions can be used in a dental product, such as a dental gel, gel sponge, or a bone cement, for other uses, including therapeutic uses, such as, but not limited to preventing or treating a jaw-bone related syndrome or disease, promoting wound healing of gum or enamel, and enhanced bone mineralization.

### CROSS-REFERENCE TO RELATED APPLICATIONS

This application claims priority to U.S. Provisional Application Ser. No. 63/376,807, filed Sep. 23, 2022, the disclosure of which is hereby incorporated herein by reference in its entirety.

### FIELD

The present embodiments relate to oral care compositions comprising geranylgeraniol and to methods of using and of making said compositions.

### BACKGROUND

The oral cavity is estimated to contain over 700 microbial species, with one study estimating the presence of over 19,000 phylotypes. One of the most prominent group of microbes in the oral cavity are streptococci, of which S. mutans is commonly associated with caries and plaque development, while S. sanguinis and S. gordonii can cause infective endocarditis when disseminated through the blood stream. S. sobrinus was also shown to have cariogenic potential. Actinomyces are found in dental biofilms and are related to periodontal disease and root caries, while lactobacilli are frequently detected in cariogenic biofilms. Of the spirochetes, T. denticola and P. gingivalis have been associated with periodontal pathogenesis. Aggregatibacter actinomycetemcomitans is a facultatively anaerobic microbe that is involved in the pathology of often severe periodontitis. E. faecalis is the most common pathogen associated with post-endodontic therapy pain and infection and is highly resistant to disinfecting agents. Candida albicans is found in root carious lesions and promotes tooth decay by inducing oral microbial dysbiosis. Lactic acid is indelibly linked to caries progression.

Oil pulling, the process of swishing oil in the oral cavity to achieve health benefits, has been used for centuries to ward off dental ails and associated systemic diseases. The practice employs the use of edible oils, which are traditionally derived from sunflower, sesame, or coconut. Lauric acid, composing nearly 50% of coconut oil, was found to have the greatest antimicrobial activity among saturated fatty acids. Medium chain triglycerides, which are composed of a large amount of capric acid with broad antimicrobial activity against oral microorganisms, present another good option for oil pulling.

Geranylgeraniol (GG) is a diterpene alcohol produced by both plants and mammals and is a crucial intermediate in the center of the mevalonate pathway. Within this biological pathway, GG is responsible for the production of carotenoids, plant steroids, vitamins and chlorophylls in plants, and the synthesis of proteins, coenzyme Q10, vitamin K, heme, and steroid hormones in mammals. Research has shown that geranylgeraniol has potential antimicrobial activity against Staphylococcus aureus and Mycobacterium tuberculosis. However, limited research has been conducted to examine other antimicrobial benefits of geranylgeraniol or against oral pathogens. Thus, there exists a need for oral care compositions comprising GG and orally acceptable carriers as an oral care product or dental product for oral hygiene or prophylactically or therapeutically treating the surfaces of the oral cavity. The oral care compositions comprising GG can be used in many aspects of dental health, including antimicrobial benefits, gum support, enhanced bone mineralization, and wound healing.

### SUMMARY

The present disclosure provides oral care compositions compatible for human administration which address the unmet needs described above. In exemplary aspects, the oral care composition of the present disclosure comprises geranylgeraniol (GG) and an orally acceptable carrier. In certain embodiments, the composition comprises GG, an orally acceptable carrier, and an antioxidant. In certain embodiments, the composition comprises GG, an orally acceptable carrier, an antioxidant, and a protease. In certain embodiments, the composition comprises GG, an orally acceptable carrier, an antioxidant, a protease, and a terpene essential oil. In certain embodiments, the composition comprises GG, an orally acceptable carrier, an antioxidant, a protease, and ascorbyl palmitate. In certain embodiments, the composition comprises GG, an orally acceptable carrier, an antioxidant, a protease, ascorbyl palmitate, and quillaja/yucca saponin.

In exemplary aspects, the oral care composition of the present disclosure comprises: GG, medium chain triglycerides (MCTs), Coenzyme Q₁₀ (CoQ₁₀), tocotrienol, bromelain, and terpene essential oil. In certain embodiments, the composition comprises GG, MCTs, CoQ₁₀, tocotrienol, bromelain, terpene essential oil, and ascorbyl palmitate. In certain embodiments, the composition comprises GG, MCTs, CoQ₁₀, tocotrienol, bromelain, terpene essential oil, ascorbyl palmitate, and quillaja/yucca saponin.

In some embodiments, the oral care composition is an oral rinse. In certain embodiments, the oral care composition is a toothpaste composition. In certain embodiments, the oral care composition is a tooth powder. In certain embodiments, the oral care composition is a mouth spray. In certain embodiments, the oral care composition is a chewing gum.

In exemplary aspects, the toothpaste composition of the present disclosure comprises GG and an orally acceptable carrier. In certain embodiments, the toothpaste composition comprises GG, an orally acceptable carrier, and a thickening agent. In certain embodiments, the toothpaste composition comprises GG, an orally acceptable carrier, a thickening agent, and a non-caloric sweetener. In certain embodiments, the toothpaste composition comprises GG, an orally acceptable carrier, a thickening agent, a non-caloric sweetener, and a humectant. In certain embodiments, the toothpaste composition comprises GG, an orally acceptable carrier, a thickening agent, a non-caloric sweetener, a humectant, and a foaming agent. In certain embodiments, the toothpaste composition comprises GG, an orally acceptable carrier, a thickening agent, a non-caloric sweetener, a humectant, a foaming agent, and an essential oil. In certain embodiments, the toothpaste composition comprises GG, an orally acceptable carrier, a thickening agent, a non-caloric sweetener, a humectant, a foaming agent, an essential oil, and an emulsifier. In certain embodiments, the toothpaste composition comprises GG, an orally acceptable carrier, a thickening agent, a non-caloric sweetener, a humectant, a foaming agent, an essential oil, an emulsifier, and annatto seed extract. In certain embodiments, the toothpaste composition comprises GG, an orally acceptable carrier, a thickening agent, a non-caloric sweetener, a humectant, a foaming agent, an essential oil, an emulsifier, annatto seed extract, and colloidal silver. In certain embodiments, the toothpaste composition comprises GG, an orally acceptable carrier, a thickening agent, a non-caloric sweetener, a humectant, a foaming agent, an essential oil, an emulsifier, annatto seed extract, colloidal silver, and grape seed extract. In certain embodiments, the toothpaste composition comprises GG, an orally acceptable carrier, a thickening agent, a non-caloric sweetener, a humectant, an essential oil, an emulsifier, annatto seed extract, colloidal silver, grape seed extract, and Lactobacillus paracasei.

In exemplary aspects, the toothpaste composition of the present disclosure comprises: GG, water, calcium phosphate, cellulose gum, peppermint oil, xylitol, glycerin, and quillaja/yucca saponin. In certain embodiments, the toothpaste composition comprises GG, water, calcium phosphate, cellulose gum, peppermint oil, xylitol, glycerin, quillaja/yucca saponin, and annatto seed extract. In certain embodiments, the toothpaste composition comprises GG, water, calcium phosphate, cellulose gum, peppermint oil, xylitol, glycerin, quillaja/yucca saponin, annatto seed extract, and colloidal silver. In certain embodiments, the toothpaste composition comprises GG, water, calcium phosphate, cellulose gum, peppermint oil, xylitol, glycerin, quillaja/yucca saponin, annatto seed extract, colloidal silver, and Lactobacillus paracasei.

Further provided herein are dental devices or oral care kits compatible for human administration which address the unmet needs described above. In exemplary aspects, the dental device is a dental floss, a tongue scraper, a dental pick, a mouth guard, or an orthodontic corrective device.

Further provided herein is a disposable toothbrush for use in oral hygiene. In exemplary aspects, the disposable toothbrush comprises an abrasive cleaning surface or bristles impregnated with any of the compositions disclosed herein, e.g., impregnated with a toothpaste composition disclosed herein.

Further provided herein are dental products compatible for human administration which address the unmet needs described above. In exemplary aspects, the dental product is a dental cement, a bone cement, a dental bonding agent, a dental porcelain powder, a pulp capping material, a retrograde filling material, a root canal filling material, or a composite resin filling material, comprising any of the compositions disclosed herein.

Further provided herein are methods of: (a) promoting gum growth, renewal, wound healing, or tissue repair; (b) removing gum adhesives; (c) preventing gum decay; (d) maintaining gum energetics; (c) improving oral health; (f) reducing plaque accumulation; (g) reducing or inhibiting gingivitis; (h) inhibiting or disrupting microbial biofilm formation in the oral cavity; (i) reducing or inhibiting formation of dental caries; (j) reducing levels of periodontopathogens; (k) reducing levels of anacrobes; (l) reducing levels of acid producing bacteria; (m) increasing relative levels of arginolytic bacteria; (n) immunizing the teeth against cariogenic bacteria; (o) reducing levels of lactic acid; (p) promoting salivary production; (q) treating, relieving, or reducing dry mouth; (r) reducing, repairing, or inhibiting early enamel lesions; (s) promoting dentin regeneration; (t) reducing or inhibiting demineralization or promoting remineralization of the teeth; (u) reducing hypersensitivity of the teeth; (v) reducing periodontal pocket depth; (w) reducing or treating bleeding on dental probing; (x) reducing or inhibiting clinical attachment loss and improving clinical attachment; (y) reducing halitosis; (z) reducing tooth staining; (aa) enhancing salivary CoQ₁₀; (bb) promoting healing of sores or cuts in the mouth or promoting wound healing of gum or enamel; (cc) cleaning the teeth and oral cavity; (dd) promoting systemic health, including cardiovascular health; (ee) preventing or restoring buccal root death or gum death; or (ff) reducing severity of gingival inflammation, comprising applying any of the compositions disclosed herein to the gum, tooth, tooth tissue, enamel, oral cavity, or tooth socket, e.g., by applying a composition disclosed herein to the oral cavity of a patient in need thereof.

Further provided herein are methods of: (a) preventing or treating a jaw-bone related syndrome or disease; (b) promoting wound healing of the gum or enamel; (c) restoring the root canal; (d) preventing or treating dental caries, root caries, tooth fracture, root fracture, cervical abrasion, tooth wearing, or gum or enamel loss; or (e) preventing or treating dry socket (alveolar osteitis), comprising applying a dental product disclosed herein to the gum, tooth tissue, enamel, oral cavity, or tooth socket of the subject, e.g., by applying a dental product disclosed herein to tooth socket of a patient in need thereof.

Further provided herein are processes of making any of the oral care compositions disclosed herein.

### BRIEF DESCRIPTION OF THE DRAWINGS

FIG. 1 shows the effects of geranylgeraniol (GG) and bisphosphonates (BP) on Alveolar Bone (AB) turnover and necrosis.

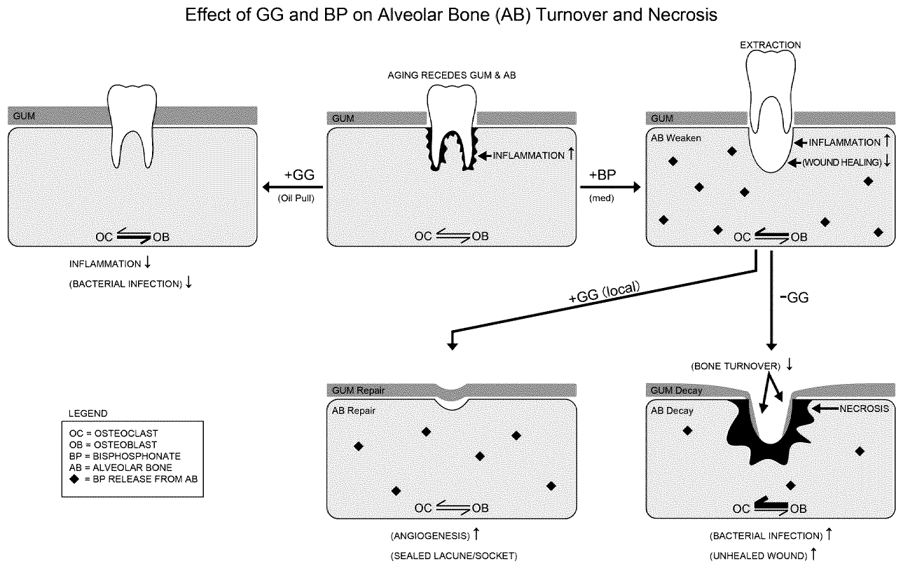

### DETAILED DESCRIPTION

### Definitions

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the term “animal” includes, but is not limited to, humans and non-human vertebrates such as wild, domestic, and farm animals.

As used herein, the term “bisphosphonate-related osteonecrosis of the jaw (BRONJ)” includes, but is not limited to, “medically-related osteonecrosis of the jaw (MRONJ),” “drug-related osteonecrosis of the jaw (DRONJ),” “age-related osteonecrosis of the jaw (ARONJ),” and “chronically-related osteonecrosis of the jaw (CRONJ).”

As used herein, the term “capric acid” (C10) refers to decanoic acid or decylic acid with the structural formula CH₃(CH₂)₈COOH.

As used herein, the term “caprylic acid” (C8) refers to octanoic acid with the structural formula CH₃(CH₂)₆CO₂H.

As used herein, the term “Coenzyme Q₁₀” or “CoQ₁₀” refers to ubiquinol, ubisemiquinone, or ubiquinone.

As used herein, the term “menaquinone” refers to vitamin K2 with the number of isoprenyl units in the side chain ranging from 4 to 13. In some embodiments, menaquinone is an antioxidant. In some embodiments, menaquinone comprises MK-4, MK-7, MK-8, MK-9, MK-10, MK-11, MK-12, MK-13, or any combinations thereof. In some embodiments, menaquinone comprises MK-4, MK-7, or a combination thereof.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system.

As used herein, the term “dentifrice” refers to products which remain in the mouth for a relatively short period of time, in which they are intimately contacted with substantially all surfaces of the teeth, and are then removed and/or systemically dissolved. Non-limiting examples of such products include toothpastes, prophylactic pastes, tooth polishes, implantable protein or collagen materials such as gels or gel sponges, implantable mineral phosphate matrices and other related professional products, as well as mouth washes, sprays, mouth rinses, dental flosses, chewing gums, lozenges, tablets, edible food products, and the like.

As used herein, the term “gel sponge” refers to absorbable gelatin sponges.

As used herein, the term “geranylgeraniol” or “GG” refers to

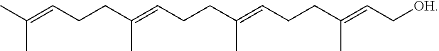 [SMILES: CC(C)=CCC/C(C)=C/CC/C(C)=C/CC/C(C)=C/CO]

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “lauric acid” (C12) refers to dodecanoic acid with the structural formula CH₃(CH₂)₁₀COOH.

As used herein, the term “mammal” means a rodent (i.e., a mouse, a rat, or a guinea pig), a monkey, a cat, a dog, a cow, a horse, a pig, or a human. In some embodiments, the mammal is a human.

The term “medium chain triglyceride” or “MCT” means any glycerol molecule ester-linked to three fatty acid molecules, each fatty acid molecule having 6-12 carbons. MCTs may be represented by the following general formula (Formula 1):

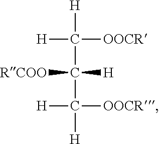

where R′, R″ and R′″ are fatty acids having 6-12 carbons in the carbon backbone esterified to a glycerol backbone. The MCTs, as provided for herein, may be prepared by any process known in the art, such as direct esterification, rearrangement, fractionation, transesterification, or the like. For example, the MCTs may be prepared by the rearrangement of a vegetable oil such as coconut oil. The length and distribution of the chain length may vary depending on the source oil. For example, MCTs containing 1-10% C6, 30-60% C8, 30-60% C10, 1-10% C12 are commonly derived from palm and coconut oils. MCTs containing greater than about 95% C8 at R′, R″ and R′″ can be made by semi-synthetic esterification of octanoic acid to glycerin. Also useful herein are mixtures comprising MCTs with about 50% total C8 and/or about 50% total C10. Commercial sources for the foregoing MCT compositions are available and known to the skilled artisan. Such MCTs behave similarly and are encompassed within the term MCTs as used herein. In some embodiments, the MCTs include coconut oil, palm kernel oil, or the combination thereof.

As used herein, the “short chain triglyceride” or “SCT” means any glycerol molecule ester-linked to three fatty acid molecules, each fatty acid molecule having 2-5 carbons. In certain embodiments, the short chain triglycerides include triacetin (glyceryl triacetate or GTA), glyceryl tripropionate (GTP), or glyceryl tributyrate (GTB).

As used herein, the “long chain triglyceride” or “LCT” means any glycerol molecule ester-linked to three fatty acid molecules, each fatty acid molecule having 14-18 carbons. In some embodiments, long chain triglycerides include: saturated long-chain fats from dairy fat, peanut oil, and other vegetable oils; monounsaturated long-chain fats from most animal and vegetable oils, particularly macadamia, olive, canola, and safflower oil; polyunsaturated long-chain fats including linoleic acid, alpha-linolenic acid (ALA), arachidonic acid, eicosapentaenoic acid (EPA), and docosahexaenoic acid (DHA).

As used herein, “triacetin”, “glyceryl triacetate” or “GTA” is a SCT having the formula of C₃Hs(OCOCH₃)₃. In certain embodiments, the compositions comprise triacetin. In certain embodiments, triacetin is a humectant, an emulsifier, and/or antimicrobial agent.

As used herein, the term “oral cavity” refers to the cavity from the lips to the epiglottis. The “hard tissues” comprise tissues such as the teeth and periodontal support and the like and the “soft tissues” comprise tissues such as the gums, the tongue, the surfaces of the buccal cavity, cranio-maxillofacial region and the like. Within the scope of this application the hard and soft tissues of the oral cavity should also be considered to comprise any devices which are used therein for example dentures, partial dentures, braces, and the like.

As used herein, the term “orally acceptable carrier” means a diluent, adjuvant, or excipient which can be used to form and/or apply the present compositions to the oral cavity in a safe and effective manner. Carriers can be liquids, such as water and oils, including those of petroleum, animal, vegetable or synthetic origin, such as peanut oil, coconut oil, mineral oil, sesame oil and the like. The carriers can also be saline, gum acacia, gelatin, starch paste, talc, keratin, colloidal silica, urea, implants (protein gels and mineral salts) and the like. In addition, auxiliary, stabilizing, thickening, lubricating and coloring agents can be used.

As used herein, the term “oral composition” or “oral care composition” means a product that in the ordinary course of usage is retained in the oral cavity for a time sufficient to contact some or all of the dental surfaces and/or oral tissues for purposes of oral activity. The “oral composition” or the “oral care composition” are used interchangeably. The oral compositions, such as those provided for herein, may be in various forms including toothpaste, dentifrice, tooth gel, tooth powders, tablets, rinse, subgingival gel, foam, mousse, chewing gum, lipstick, sponge, floss, prophy paste, petrolatum gel, or denture product. The oral composition may also be incorporated onto strips or films for direct application or attachment to oral surfaces, incorporated into the bristles of a toothbrush, or incorporated into floss.

As used herein, the term “oral rinse” encompasses both pre-brushing dental rinses and mouthwashes which are generally used after brushing.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments include pharmaceutically acceptable salt of the compounds described herein. The present embodiments also include quaternary ammonium salts of the compounds described herein, where the compounds have one or more tertiary amine moiety.

As used herein, the term “purified” means that when isolated, the isolate contains at least 90%, at least 95%, at least 98%, or at least 99% of a compound described herein by weight of the isolate.

As used herein, the term “quillaja saponin” refers to purified saponin extracts from the Chilean tree Quillaja saponaria Molina. As used herein, the term “yucca saponin” refers to purified saponin extracts from yucca or cassava.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, elimination or amelioration of a disorder, or side-effects. The amount needed to elicit the therapeutic response can be determined based on the age, health, size and sex of the subject. Optimal amounts can also be determined based on monitoring of the subject's response to treatment.

As used herein, the term “toothpaste composition” means a composition that is suitable to be placed in the oral cavity and to come in contact with the teeth and/or gums.

As used herein, the terms “treat,” “treated,” or “treating” mean both therapeutic treatments wherein the object is to slow down (lessen) an undesired physiological condition, disorder or disease, or obtain beneficial or desired clinical results. As used herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

Elements of these compositions and their benefits are described in more detail below.

The oral cavity consists of a jaw with teeth and gums, and this intersection presents a ubiquitous opportunity for bacterial infection. Additionally, the turnover rate of the mandible alveolar bones—also part of the jaw—is very rapid, about 10-20× faster than other bones. In the event of a local trauma like a tooth extraction, the alveolar bone repairs to seal the empty socket (lacune) and remodels the jaw bone rapidly, thus managing inflammation, wound healing, gum growth and infection. Interruptions to such a rapid bone remodeling will bring sequelae of oral morphologic dysfunctions, resulting in a loss of jaw bone repair.

Drugs are known to disrupt jaw bone remodeling, particularly bisphosphonates (anti-osteoporosis and anti-metastatic medications) and statins (anti-lipidemia medications). These medications systemically and biochemically inhibit endogenous GG with pathological consequences. Gum healing and jaw bone repair are two compelling indications for GG treatment in tooth extraction—or otherwise jaw damage—in mammals on bisphosphonates.

### Geranylgeraniol

Staphylococcus aureus is a bacterium frequently found on the skin and in the upper respiratory tract, and has also been recognized as a constituent of the oral flora. Although typically regarded as a commensal member of the microbiota, S. aureus can cause opportunistic infections, and 10% of oral isolates of the bacterium were found to be methicillin-resistant. One study examining GG's antibacterial activity found that the diterpene inhibited growth of S. aureus by damaging bacterial cell membranes, with the caveat that smaller concentrations were more effective than higher concentrations. GG's optimum bactericidal effect was achieved by applying 0.15-1.25 ug/ml, which decreased viable bacterial cell count by a factor of two within two hours, and more with longer exposure.

As opposed to S. aureus infections, oral tuberculosis, caused by Mycobacterium tuberculosis, is a much rarer occurrence, albeit important in early detection to limit mortality. Among terpene compounds, GG was identified to be a potent inhibitor of M. tuberculosis. At 6.25 ug/ml, GG resulted in 96% inhibition of the bacterium, the effect thought to be based on terpenoid chain length.

Beyond oral microbes, GG has also been shown to inhibit parasitic Leishmaniasis, while a derivative of GG—geranylgeranylacetone—was able to suppress influenza virus infection.

Providing oral health benefits from an entirely different dimension, GG was found to directly benefit gums, bone mineralization, and wound healing. The majority of these studies were conducted in models of bisphosphonate-induced osteonecrosis of the jaw (BRONJ). Nitrogen-containing bisphosphonates, such as zoledronic acid, are used extensively in the treatment of osteoporosis, where bisphosphonates target enzymes in the mevalonate pathway that play a rate-limiting role in bone resorption. Ironically, while preserving bone in the rest of the body, the lack of bone turnover and capillary maintenance with bisphosphonates in the jaw—which has a higher remodeling rate—causes altered wound healing, chronic infections, and osteonecrosis.

Helping gingival and osteocyte cells survive, GG has been shown to improve gingiva and alveolar bone morphology. In one study, GG significantly increased the viability of gingival epithelial cells that had been treated with bisphosphonates, while also improving the cell morphology of both gingival epithelial and fibroblast cells. GG further reversed cellular effects of bisphosphonates in oral gingival fibroblasts by restoring cell proliferation, migration, and adhesion defects.

Biomineralization is a dynamic process, with teeth being at particular risk of demineralization due to their anatomical arrangement and location. Further, the oral mineralization process can be impacted by drugs such as bisphosphonates, which have been shown to suppress mesenchymal stem cell and osteoblast viability and proliferation, ultimately leading to the death of bone tissue. Whereas treatment with bisphosphonates dramatically decreased viable mesenchymal stem cells by inhibiting geranylgeranylation, GG preserved these important differentiator cells, reversing its apoptosis and cell cycle arrest via rescuing activity of the Rho-dependent YAP activation pathway. Timing of GG addition was an important consideration: It was best to apply GG during the early stages of medication (within 7 days of bisphosphonate treatment and/or tooth extraction) to maximize GG's ability to rescue cell viability and increase total protein in cells. On a genetic level, GG was able to upregulate expression of collagen type I and osteopontin (required for osteoblast differentiation), as well as vascular endothelial growth factors, which are necessary for bone healing and increased bone formation. Similar research concurs that GG benefits bone turnover via angiogenic gene expression regulation. Another mechanism by which GG aids bone mineralization is by inhibiting the formation of aberrant and hypernucleated osteoclasts, cells responsible for the dissolution and absorption of bone. This inhibition is accomplished via suppression of receptor activator of NF-kB ligand (RANKL) expression and abolished disruption of osteoclastic actin rings.

Wound healing after periodontal surgery carries a notable risk of gum degradation, and the bone remodeling process continues 3-6 months following extraction. Adding to that, bisphosphonates can contribute severe wound healing problems, with clinical manifestations including ulcers, reopening of the wound, and bone necrosis. Systemic administration of GG decreased apoptosis of osteocytes—the longest-living type of bone cells—and improved bone healing of extraction sockets in animals with BRONJ. GG aided healing of extraction sockets by preventing osteonecrosis and increasing the amount of connective tissue, angiogenic blood vessels and cells collectively referred to as dental pulp. Restored bone healing and bone mineralization was visualized via micro-CT scan in this animal model. In a separate preclinical study, rats were treated with bisphosphonates for three weeks prior to extraction of first molars. Once the molars were removed, one group of animals was treated with a GG solution applied daily into the extraction socket, which significantly improved wound healing and tissue proliferation compared to the untreated group. Whereas animals treated with bisphosphonates alone showed an 80% progression towards osteonecrosis, animals treated with both bisphosphonates and GG had a significantly improved gum and extracted socket healing by about 45-67%. GG had an additional positive influence on the wound healing process by decreasing inflammation scores, infection, and exposed bone. Accordingly, GG covers many aspects of dental health, including antimicrobial benefits, gum support, enhanced bone mineralization, and wound healing.

### Pulling Oil

The use of sesame and coconut oils was as effective as the prescription germicidal mouthwash chlorhexidine in reducing several parameters related to plaque-induced gingivitis. These pulling oils were especially potent in reducing levels of the anaerobic bacterium Streptococcus mutans, which is the most common organism to cause caries. In 20 subjects tested with sesame oil or chlorhexidine for up to two weeks, both approaches lowered levels of S. mutans in plaque and saliva samples. Similarly, a comparison study of coconut oil versus chlorhexidine given to 50 subjects over 30 days showed equally significant decreases in S. mutans. Compared to a placebo of distilled water, patients treated with either coconut oil or chlorhexidine carried significantly lower levels of S. mutans in their saliva, with coconut oil and chlorhexidine producing similar results. Another study comparing the effects of sesame oil and chlorhexidine in 20 adolescent boys with plaque-induced gingivitis showed that after 10 days of treatment, both pulling oil and mouthwash effectively reduced colony counts of aerobic microorganisms, while also lowering plaque and modified gingival index scores.

Halitosis—bad breath—reflects complex interactions between several oral bacterial species, arising as a consequence of microbial putrefaction of food, debris, cells, saliva, and blood. In comparison to prescription mouthwash, oil pulling was equally effective in reducing oral malodor. In one randomized controlled trial, 60 subjects were given either sesame oil, chlorhexidine, or a control for 3 weeks. Both sesame oil and chlorhexidine reduced not only plaque index, gingival index, and anaerobic bacterial counts, but also objective and subjective organoleptic scores of oral malodor. The authors noted that sesame oil was as effective as chlorhexidine in reducing halitosis and the microbes causing it. A separate pilot study similarly concluded that oil pulling was as effective as the use of chlorhexidine on halitosis, where both reduced plaque and gingival indices along with organoleptic breath and self-assessment. Furthermore, OTC chlorhexidine mouthwash—used 2 or more times a day—increased pre-diabetes/diabetes by 55% (Joshipura K J et al, 2017) and hypertension by 12% (Joshipura K et al, 2020).

Pulling oil, regardless of oil origin, has a long history of use, with successful clinical studies equating its efficacy to prescription chemical mouthwashes in addition to further oral health benefits.

### Coenzyme Q10

Scientists at Liverpool's Morton laboratory caught a first glimpse at ubiquinone in 1955, but it was not until 1957 that Frederick Crane famously isolated CoQ10 from beef hearts. The discovery launched an investigation into one of today's most well-known and widely-studied nutrients.

Since then, researchers have found that CoQ10 declines with age, and coincides with a variety of chronic conditions, including heart disease, type 2 diabetes, and cancer. Research suggests a link between periodontitis and cardiovascular disease, and CoQ10 may play a specific role: Plasma samples taken from patients with both periodontitis and coronary heart disease showed significantly lower CoQ10 levels. Further, low levels of CoQ10 and high levels of ROS production were seen in periodontitis patients, where mitochondrial dysfunction was promoted by the microbe Porphyromonas gingivalis, causing oxidative stress and altered cytokine homeostasis.

Evidence of a CoQ10 deficiency in gingiva was first shown by a team of researchers led by CoQ10 pioneer Karl Folkers, who determined the structure of the enzyme in 1958. The researchers found that adjunctive treatment of oral CoQ10 in periodontal therapy significantly improved periodontal scores and decreased periodontal pocket depth, while promoting healing of gingival biopsy sites. Folkers and his team were the first to recommend CoQ10 as adjunctive treatment with current dental practices for periodontitis.

Today, CoQ10 is used by some dentists and periodontists, although the practice is far from routine. The majority of applications are performed topically and in combination with traditional scaling and root planing for periodontitis. Several smaller studies, enrolling between 10-20 patients, found that CoQ10 was a useful adjunct to periodontal therapy. One study in patients with chronic periodontitis tested CoQ10's effect on superoxide dismutase in gingival crevicular fluid, which showed significant improvements after 3 months, suggesting that CoQ10 can boost the antioxidant concentration. Another study utilized both topical and intrasulcular application of a CoQ10 gel in combination with scaling and root planing, which resulted in significant improvements in plaque index, gingival bleeding index, and probing pocket depth. To determine the differences between extrasulcular topical and intrapocket application of CoQ10, one study compared these approaches to the use of scaling and root planing alone. While all treatment groups showed improvement in plaque, gingival, and bleeding index along with probing pocket depth, only the intrapocket gel application in combination with scaling and root planing showed a significant reduction compared to the use of intrapocket gel alone, leading researchers to believe that CoQ10 gel has an additive effect to scaling and root planing alone. A similar study that tested the effects of topical CoQ10 on adult periodontitis showed that only sites treated with CoQ10 (following scaling and root planing) presented with improved gingival index, bleeding on probing, and peptidase activity derived from periodontopathic bacteria.

Two slightly larger randomized placebo-controlled trials studying CoQ10's anti-gingivitis effects advocated for the use of CoQ10 as an adjunct to traditional scaling and root planing. One of these trials administered CoQ10 orally for up to 3 months, which resulted in a significant decrease in gingival inflammation compared to the mechanical debridement method alone. The other trial utilized CoQ10 topically, and showed promising results with both CoQ10 use alone as well as in combination with scaling and root planing.

Specialized formulations or routes of administration have also been shown advantageous for the use of CoQ10 in periodontal disease. One group created a CoQ10 nanoparticle system, which—in combination with scaling and root planing—resulted in significant management of periodontal parameters and was better than scaling and root planing alone all the while enhancing antioxidant activity. Another group delivered CoQ10 subgingivally for sustained release in smokers with chronic periodontitis, and noted that plaque index, modified sulcular bleeding index, probing pocket depth and clinical attachment level all improved after one and 3 months.

Combining CoQ10 with other dietary ingredients such as vitamin E successfully improved periodontitis parameters, while CoQ10 use was comparable to the use of tea tree oil and 0.8% hyaluronic acid in the treatment of periodontitis when administered together with scaling and root planing.

Beyond aiding periodontitis treatment, use of oral CoQ10 had significant benefits for salivary secretions, suggesting its ability to reduce dry mouth and halitosis. In a study of 66 patients, 100 mg per day of CoQ10 (given as either ubiquinone or ubiquinol) significantly improved salivary secretions and salivary CoQ10 content, hence attenuating dry mouth symptoms. In another study, 40 subjects with persistent mouth dryness were given gummy candies containing 50 mg ubiquinol twice daily for 8 weeks, which resulted not only in significantly increased CoQ10 levels in saliva, but also increased saliva flow rate while improving fatigue and mouth dryness. The authors believe that CoQ10's enhanced ATP production and suppressed oxidative stress are responsible for the observed benefits.

In animal studies, CoQ10 topical application was tested in rats following tooth extraction, and was shown to significantly accelerate wound healing. The improved healing was attributed to higher collagen density and lower inflammatory biomarkers within the first few days of application.

### Bromelain

Bromelain belongs to a group of protein-digesting enzymes, and boasts fibrinolytic, anti-thrombotic, anti-edematous and anti-inflammatory properties. These unique characteristics of the proteolytic enzyme contribute to its manifold benefits for oral health that are not only limited to antibacterial and anti-plaque and -gingivitis activities, but also include stain removal and improved wound healing following dental work such as extraction.

One clinical study aimed to examine the efficacy of bromelain in controlling edema and related pain in the inflamed area where a third molar extraction was performed. In the study, patients were either treated with an antibacterial solution containing 40 mg bromelain, which was administered every 6 hours for 6 days, or the same antibacterial solution with nonsteroidal inflammatory drug ketoprofen. Results demonstrated the effectiveness of bromelain in treating postoperative swelling following tooth extraction, and confirmed that bromelain was equally effective in reducing pain and edema when compared to ketoprofen. Bromelain in combination with trypsin and rutin had a similar beneficial effect in patients that had an impacted third molar removed. The clinical trial of 64 patients showed that the proteolytic enzyme mixture was more potent in facilitating wound healing than the commonly used serratiopeptidase.

Bromelain was not only helpful when used as a topical in tooth extractions, but also in the form of an oral supplement. Patients that took the bromelain registered dose of 1,000 FIP following wisdom tooth extraction saw significant reductions in post-op swelling and inflammation compared to a placebo. In combination with amoxicillin, bromelain was also shown to be effective in patients following third molar removal when taken at 200 mg two times per day for 5 days, reducing swelling and pain in up to 70% of study subjects. In a separate double-blind placebo controlled trial, patients with gingival regression that underwent gum grafting were given a daily dose of 500 mg bromelain for 10 days, and noticed a significant reduction in pain at the graft donor site along with improved epithelialization indicative of wound healing. In vitro, the regenerative properties of bromelain were tested on mesenchymal stem cells. When used in combination with dexamethasone sodium phosphate, bromelain significantly activated the stem cells while increasing hyaluronan and collagen production and anti-inflammatory cytokine release. Bromelain-mediated inhibition of inflammatory cytokines appears to be due to a decrease in the NFkB and MAPK pathways, where bromelain decreased several interleukins and adhesion molecules, but also promoted mineralization in human dental pulp cells. Collectively, these studies suggest bromelain's potential for use in regenerative endodontics and as an adjuvant following dental extractions.

Commercial mouth rinses are designed to fight caries and plaque build-up, but some of these products carry undesirable side effects, including staining. Bromelain's stain removal efficacy has been evaluated in several studies. In human molar teeth, the effects of a bromelain/papain toothpaste were compared to a standard dentifrice, with bromelain and papain resulting in significantly greater stain removal based on lightness values. When applied to the enamel of bovine tooth samples that had been stained in coffee solution, bromelain gel caused a significant color change compared to the negative control, suggesting that the enzyme gel could be a good stain removal alternative to agents containing hydrogen or carbamide peroxide. Clinically, two studies examined the efficacy of a bromelain-containing enzymatic toothpaste in comparison to Colgate. In one, tooth lightness and stain removal were significantly higher for the test toothpaste, and were attributed to the role of proteolytic enzymes. A second study assessed not only the effectiveness, but also stain prevention efficacy of a bromelain-containing versus an abrasive toothpaste. Both abrasive and enzymatic toothpastes reduced staining after one month, but only the enzymatic toothpaste showed significant stain reductions after two months of use, suggesting a stain prevention mechanism. The researchers concluded that enzymatic toothpastes may be preferable, since abrasive toothpastes can cause dental wear.

Surprisingly, one of bromelain's manifold dental applications relates to improving bond strength for dental fillings. Studies involving bromelain as a deproteinizing agent in human molars or premolars show that when bromelain was applied (instead of standard sodium hypochlorite), shear bond strength was maximized, which is thought to be due to bromelain's ability to remove unsupported collagen fiber. One problem with dental fillings that increases with bleaching is microleakage, where bacteria, oral fluids, ions and other molecules diffuse the tooth and filling material interface. Applying a 10% bromelain enzyme solution to premolars following filling with composite resin and bleaching, on the other hand, significantly reduced microleakage.

Dental caries is the predominant cause of tooth loss in children and young adults, and was—as early as the 16th century when Leeuwenhoek first saw plaque bacteria under the microscope—suspected to be caused by microorganisms. In children with dental anxiety, removal of caries by purely mechanical means is a challenge, and hence minimally invasive alternatives are sought after. The efficacy of bromelain and papain gels for caries removal was evaluated in primary molars with cavities extending into the dentin, and bromelain was found to be superior in the amount of caries removed. Moreover, bromelain applied for caries removal resulted in microhardness levels that were comparable to dentin, suggesting that this less invasive method of chemomechanical caries removal may be preferable over traditional approaches. Bromelain, at a dilution of 2 mg/ml, was also effective against Streptococcus mutans, a bacterial strain that is not only involved in caries development, but also in periodontitis, and was found to exert antibacterial effects against Porphyromonas gingivalis at a dilution of 4.15 mg/ml. Anti-plaque fighting benefits for bromelain are further supported by a clinical trial testing a bromelain-containing enzymatic toothpaste in comparison to Colgate, where the test dentifrice resulted in a significantly lower plaque and gingival index than the standard fluoride toothpaste.

Bromelain has a broad range of dental health applications, ranging from caries reduction to tooth whitening and extending into targeted approaches for dental fillings and extractions.

### Oral Care Compositions

Embodiments of various compounds and salts thereof are provided. Where a variable is not specifically recited, the variable can be any option described herein, except as otherwise noted or dictated by context.

In exemplary aspects, the oral care composition of the present disclosure comprises geranylgeraniol (GG) and an orally acceptable carrier. In certain embodiments, the composition comprises GG, an orally acceptable carrier, and an antioxidant. In certain embodiments, the composition comprises GG, an orally acceptable carrier, an antioxidant, and a protease. In certain embodiments, the composition comprises GG, an orally acceptable carrier, an antioxidant, a protease, and a terpene essential oil. In certain embodiments, the composition comprises GG, an orally acceptable carrier, an antioxidant, a protease, and ascorbyl palmitate. In certain embodiments, the composition comprises GG, an orally acceptable carrier, an antioxidant, a protease, ascorbyl palmitate, and quillaja/yucca saponin.

In exemplary aspects, the oral care composition of the present disclosure comprises: GG, medium chain triglycerides (MCTs), Coenzyme Q₁₀ (CoQ₁₀), tocotrienol, bromelain, and terpene essential oil. In certain embodiments, the composition comprises GG, MCTs, CoQ₁₀, tocotrienol, bromelain, terpene essential oil, and ascorbyl palmitate. In certain embodiments, the composition comprises GG, MCTs, CoQ₁₀, tocotrienol, bromelain, terpene essential oil, ascorbyl palmitate, and quillaja/yucca saponin.

In some embodiments, the oral care composition is an oral rinse. In certain embodiments, the oral care composition is a toothpaste composition. In certain embodiments, the oral care composition is a tooth powder. In certain embodiments, the oral care composition is a mouth spray. In certain embodiments, the oral care composition is a chewing gum.

In exemplary aspects, the toothpaste composition of the present disclosure comprises GG and an orally acceptable carrier. In certain embodiments, the toothpaste composition comprises GG, an orally acceptable carrier, and a thickening agent. In certain embodiments, the toothpaste composition comprises GG, an orally acceptable carrier, a thickening agent, and a non-caloric sweetener. In certain embodiments, the toothpaste composition comprises GG, an orally acceptable carrier, a thickening agent, a non-caloric sweetener, and a humectant. In certain embodiments, the toothpaste composition comprises GG, an orally acceptable carrier, a thickening agent, a non-caloric sweetener, a humectant, and a foaming agent. In certain embodiments, the toothpaste composition comprises GG, an orally acceptable carrier, a thickening agent, a non-caloric sweetener, a humectant, a foaming agent, and an essential oil. In certain embodiments, the toothpaste composition comprises GG, an orally acceptable carrier, a thickening agent, a non-caloric sweetener, a humectant, a foaming agent, an essential oil, and an emulsifier. In certain embodiments, the toothpaste composition comprises GG, an orally acceptable carrier, a thickening agent, a non-caloric sweetener, a humectant, a foaming agent, an essential oil, an emulsifier, and annatto seed extract. In certain embodiments, the toothpaste composition comprises GG, an orally acceptable carrier, a thickening agent, a non-caloric sweetener, a humectant, a foaming agent, an essential oil, an emulsifier, annatto seed extract, and colloidal silver. In certain embodiments, the toothpaste composition comprises GG, an orally acceptable carrier, a thickening agent, a non-caloric sweetener, a humectant, a foaming agent, an essential oil, an emulsifier, annatto seed extract, colloidal silver, and grape seed extract. In certain embodiments, the toothpaste composition comprises GG, an orally acceptable carrier, a thickening agent, a non-caloric sweetener, a humectant, a foaming agent, an essential oil, an emulsifier, annatto seed extract, colloidal silver, grape seed extract, and Lactobacillus paracasei.

In exemplary aspects, the toothpaste composition of the present disclosure comprises: GG, water, calcium phosphate, cellulose gum, peppermint oil, xylitol, glycerin, and quillaja/yucca saponin. In certain embodiments, the toothpaste composition comprises GG, water, calcium phosphate, cellulose gum, peppermint oil, xylitol, glycerin, quillaja/yucca saponin, and annatto seed extract. In certain embodiments, the toothpaste composition comprises GG, water, calcium phosphate, cellulose gum, peppermint oil, xylitol, glycerin, quillaja/yucca saponin, annatto seed extract, and colloidal silver. In certain embodiments, the toothpaste composition comprises GG, water, calcium phosphate, cellulose gum, peppermint oil, xylitol, glycerin, quillaja/yucca saponin, annatto seed extract, colloidal silver, and Lactobacillus paracasei.

In some embodiments, the oral care composition of the present disclosure comprises about 0.05% to about 8%, about 0.1% to about 5%, about 0.2% to about 4%, about 0.5% to about 2%, about 0.6% to about 1.5%, about 0.75% to about 1.2%, or about 0.75% to about 1% w/w GG. In some embodiments, the oral care composition comprises about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w GG. In certain embodiments, the oral care composition comprises about 0.75% GG.

In some embodiments, the oral care composition of the present disclosure comprises about 1% to about 99%, about 5% to about 99%, about 10% to about 99%, about 20% to about 99%, about 30% to about 99%, about 40% to about 99%, about 50% to about 99%, about 60% to about 99%, about 70% to about 99%, about 80% to about 99%, or about 90 to about 99% orally acceptable carrier. In some embodiments, the oral care composition comprises about 1%, about 5%, about 10%, about 20%, about 30%, about 40%, about 50%, about 60%, about 70%, about 75%, about 80%, about 85%, about 90%, about 91%, about 92%, about 93%, about 94%, about 95%, about 96%, about 97%, about 98%, or about 99% orally acceptable carrier.

Orally acceptable carriers are well known in the art, and include without limitation, medium chain triglycerides (MCTs), short chain triglycerides, long chain triglycerides, coconut oil, palm kernel oil, sesame oil, sunflower oil, or any combination thereof. In certain embodiments, the orally acceptable carrier is medium chain triglycerides. In some embodiments, the medium chain triglycerides consist of aliphatic tails with about 6 to about 12 carbon atoms. In certain embodiments, the orally acceptable carrier is short chain triglycerides. In some embodiments, the short chain triglycerides consist of aliphatic tails with about 2 to about 5 carbon atoms. In certain embodiments, the short chain triglycerides include triacetin (glyceryl triacetate or GTA), glyceryl tripropionate (GTP), or glyceryl tributyrate (GTB). In certain embodiments, the orally acceptable carrier is long chain triglycerides. In some embodiments, the long chain triglycerides consist of aliphatic tails with about 14 to about 18 carbon atoms. In some embodiments, long chain triglycerides include: saturated long-chain fats from dairy fat, peanut oil, and other vegetable oils; monounsaturated long-chain fats from most animal and vegetable oils, particularly macadamia, olive, canola, and safflower oil; polyunsaturated long-chain fats including linoleic acid, alpha-linolenic acid (ALA), arachidonic acid, eicosapentaenoic acid (EPA), and docosahexaenoic acid (DHA).

In some embodiments, the medium chain triglycerides comprise caprylic acid, capric acid, lauric acid, or any combination thereof. In some embodiments, the medium chain triglycerides comprise capyrylic acid, capric acid, and lauric acid, wherein the weight ratio of caprylic acid, capric acid, and lauric acid is about 4:3:3.

In some embodiments, the oral care composition of the present disclosure comprises about 1% to about 99%, about 5% to about 99%, about 10% to about 99%, about 20% to about 99%, about 30% to about 99%, about 40% to about 99%, about 50% to about 99%, about 60% to about 99%, about 70% to about 99%, about 80% to about 99%, or about 90 to about 99% of medium chain triglycerides. In some embodiments, the oral care composition comprises about 1%, about 5%, about 10%, about 20%, about 30%, about 40%, about 50%, about 60%, about 70%, about 75%, about 80%, about 85%, about 90%, about 91%, about 92%, about 93%, about 94%, about 95%, about 96%, about 97%, about 98%, or about 99% medium chain triglycerides. In certain embodiments, the oral care composition comprises about 97% medium chain triglycerides.

In some embodiments, the oral care composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w of an antioxidant. In some embodiments, the oral care composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w of an antioxidant.

Antioxidants are well known in the art, and include without limitation, Coenzyme Q₁₀ (Co Q₁₀, as ubiquinone, semiquinone, ubiquinol or the combination thereof), tocotrienol, ascorbyl palmitate, α-hydroxy acids (for example citric acid, lactic acid, malic acid), humic acid, bile acid, bile extracts, bilirubin, biliverdin, EDTA, EGTA and derivatives thereof, vitamin C and derivatives (for example ascorbyl palmitate, magnesium ascorbyl phosphate, ascorbyl acetate), tocopherols and derivatives, and tocotrienols and derivatives.

In some embodiments, the oral care composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w CoQ₁₀. In some embodiments, the oral care composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w CoQ₁₀. In certain embodiments, the oral care composition comprises about 0.05% CoQ₁₀.

In some embodiments, the oral care composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w tocotrienol. In some embodiments, the oral care composition comprises about 0.001%, about 0.0025%, about 0.005%, about 0.0075%, about 0.01%, about 0.015%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w tocotrienol. In certain embodiments, the oral care composition comprises about 0.03% tocotrienol.

In some embodiments, the oral care composition of the present disclosure comprises about 0.01% to about 12%, about 0.05% to about 10%, about 0.1% to about 8%, about 0.5% to about 6%, about 1% to about 5%, about 2% to about 4%, about 2.5% to about 3% w/w ascorbyl palmitate. In some embodiments, the oral care composition comprises about 0.001%, about 0.005%, about 0.01%, about 0.05%, about 0.1%, about 0.5%, about 1%, about 1.25%, about 1.5%, about 1.75%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, or about 12% w/w ascorbyl palmitate.

In some embodiments, the oral care composition of the present disclosure comprises about 0.01% to about 5%, about 0.025% to about 4.5%, about 0.05% to about 5%, about 0.075% to about 3.5%, about 0.1% to about 3%, about 0.15% to about 2.5%, about 0.2% to about 2%, about 0.25% to about 1.5%, about 0.3% to about 1%, about 0.35% to about 0.95%, about 0.4% to about 0.9%, about 0.45% to about 0.85%, about 0.5% to about 0.8%, about 0.55% to about 0.75%, or about 0.6% to about 0.7% w/w of a protease. In some embodiments, the oral care composition comprises about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, or about 5% w/w of a protease.

Proteases are well known in the art, and include without limitation include bromelain, papain, pancreatin, trypsin, and chymotrypsin. In certain embodiments, the protease is bromelain.

In some embodiments, the oral care composition of the present disclosure comprises about 0.01% to about 5%, about 0.025% to about 4.5%, about 0.05% to about 5%, about 0.075% to about 3.5%, about 0.1% to about 3%, about 0.15% to about 2.5%, about 0.2% to about 2%, about 0.25% to about 1.5%, about 0.3% to about 1%, about 0.35% to about 0.95%, about 0.4% to about 0.9%, about 0.45% to about 0.85%, about 0.5% to about 0.8%, about 0.55% to about 0.75%, or about 0.6% to about 0.7% w/w bromelain. In some embodiments, the oral care composition comprises about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, or about 5% w/w bromelain. In certain embodiments, the oral care composition comprises about 0.5% bromelain.

In some embodiments, the oral care composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w of a terpene essential oil. In some embodiments, the oral care composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w of a terpene essential oil.

Terpene essential oils are well known in the art, and include without limitation, cinnamon oil, tea tree oil, lemongrass oil, clove oil, peppermint oil, spearmint oil, wintergreen oil, cedarwood oil, lemon oil, lime oil, eucalyptus oil, or oregano oil. In certain embodiments, the terpene essential oil is peppermint oil. In certain embodiments, the terpene essential oil is peppermint oil. In certain embodiments, the terpene essential oil is lemon oil.

In some embodiments, the oral care composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w peppermint oil. In some embodiments, the oral care composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w peppermint oil.

In some embodiments, the oral care composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w lemon oil. In some embodiments, the oral care composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w lemon oil.

In some embodiments, the oral care composition of the present disclosure comprises about 0.001% to about 5%, about 0.005% to about 3%, about 0.0075% to about 2%, about 0.01% to about 1.5%, about 0.05% to about 1%, about 0.075% to about 0.75%, about 0.1% to about 0.5% w/w of an emulsifier. In some embodiments, the oral care composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w of an emulsifier.

Emulsifiers are well known in the art, and include without limitation, cocamidopropyl betaine, quillaja saponin, yucca saponin, sodium coco sulfate, lecithin, gum Arabic, acetic acid esters, lactic acid esters, magnesium stearate, mono- and diglycerides, triglycerides (e.g., triacetin, glyceryl tripropionate, and glyceryl tributyrate), or sucrose esters. In certain embodiments, the emulsifier is quillaja saponin or yucca saponin.

In some embodiments, the oral care composition of the present disclosure comprises about 0.001% to about 5%, about 0.005% to about 3%, about 0.0075% to about 2%, about 0.01% to about 1.5%, about 0.05% to about 1%, about 0.075% to about 0.75%, about 0.1% to about 0.5% w/w quillaja/yucca saponin. In some embodiments, the oral care composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w quillaja/yucca saponin.

In exemplary aspects, the oral care composition of the present disclosure is a toothpaste composition. In some embodiments, the toothpaste composition comprises about 0.05% to about 8%, about 0.1% to about 5%, about 0.2% to about 4%, about 0.5% to about 2%, about 0.6% to about 1.5%, about 0.75% to about 1.2%, or about 0.75% to about 1% w/w GG. In some embodiments, the toothpaste composition comprises about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w GG. In certain embodiments, the toothpaste composition comprises about 1% GG.

In some embodiments, the toothpaste composition of the present disclosure comprises about 1% to about 60%, about 3% to about 50%, about 5% to about 40%, about 7% to about 30%, about 9% to about 20%, or about 10% to about 15% of an orally acceptable carrier. In some embodiments, the toothpaste composition comprises about 0.5%, about 1%, about 3%, about 5%, about 7%, about 9%, about 11%, about 13%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, or about 50% of an orally acceptable carrier.

In some embodiments, the toothpaste composition of the present disclosure comprises about 1% to about 60%, about 3% to about 50%, about 5% to about 40%, about 7% to about 30%, about 9% to about 20%, or about 10% to about 15% water. In some embodiments, the toothpaste composition comprises about 0.5%, about 1%, about 3%, about 5%, about 7%, about 9%, about 11%, about 13%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, or about 50% water. In certain embodiments, the toothpaste composition comprises about 25% water.

In some embodiments, the toothpaste composition of the present disclosure comprises about 1% to about 60%, about 3% to about 50%, about 5% to about 40%, about 7% to about 30%, about 9% to about 20%, or about 10% to about 15% of a non-caloric sweetener. In some embodiments, the toothpaste composition comprises about 0.5%, about 1%, about 3%, about 5%, about 7%, about 9%, about 11%, about 13%, about 15%, about 20%, about 25%, about 30%, about 35%, about 40%, about 45%, or about 50% of a non-caloric sweetener.

Non-caloric sweeteners are well known in the art and include without limitation, xylitol, sorbitol, erythritol, sucralose, rebaudioside A, or mogrosides.

In some embodiments, the toothpaste composition of the present disclosure comprises about 1% to about 60%, about 3% to about 50%, about 5% to about 40%, about 7% to about 30%, about 9% to about 20%, or about 10% to about 15% xylitol. In some embodiments, the toothpaste composition comprises about 0.5%, about 1%, about 3%, about 5%, about 7%, about 9%, about 11%, about 13%, about 15%, about 20%, about 25%, about 30%, about 35%, about 40%, about 45%, about 50%, about 55%, or about 60% xylitol. In certain embodiments, the toothpaste composition comprises about 35% xylitol.

In some embodiments, the toothpaste composition of the present disclosure comprises about 1% to about 95%, about 5% to about 85%, about 10% to about 75%, about 15% to about 65%, about 20% to about 55%, about 25% to about 45%, or about 30% to about 35% of an abrasive. In some embodiments, the toothpaste composition comprises about 0.5%, about 1%, about 5%, about 10%, about 15%, about 20%, about 25%, about 26%, about 27%, about 28%, about 29%, about 30%, about 31%, about 32%, about 33%, about 34%, about 35%, about 40%, about 45%, about 50%, about 55%, about 60%, about 65%, about 70%, about 75%, about 80%, about 85%, about 90%, or about 95% of an abrasive.

Abrasives are well known in the art and include without limitation, hydrated silica, calcium carbonate, sodium bicarbonate, bentonite clay, or calcium phosphate. In certain embodiments, the abrasive is calcium phosphate.

In some embodiments, the toothpaste composition of the present disclosure comprises about 1% to about 95%, about 5% to about 85%, about 10% to about 75%, about 15% to about 65%, about 20% to about 55%, about 25% to about 45%, or about 30% to about 35% calcium phosphate. In some embodiments, the toothpaste composition comprises about 0.5%, about 1%, about 5%, about 10%, about 15%, about 20%, about 25%, about 26%, about 27%, about 28%, about 29%, about 30%, about 31%, about 32%, about 33%, about 34%, about 35%, about 40%, about 45%, about 50%, about 55%, about 60%, about 65%, about 70%, about 75%, about 80%, about 85%, about 90%, or about 95% calcium phosphate. In certain embodiments, the toothpaste composition comprises 30% calcium phosphate.

In some embodiments, the toothpaste composition of the present disclosure comprises about 1% to about 50%, about 3% to about 40%, about 5% to about 30%, about 7% to about 25%, or about 10% to about 20% of a humectant. In some embodiments, the toothpaste composition comprises about 0.5%, about 1%, about 3%, about 5%, about 7%, about 9%, about 11%, about 13%, about 15%, about 20%, about 25%, about 30%, about 35%, about 40%, about 45%, or about 50% of a humectant.

Humectants are well known in the art and include without limitation, glycerin, sorbitol, triacetin, or propylene glycol. In certain embodiments, the humectant is glycerin.

In some embodiments, the toothpaste composition of the present disclosure comprises about 1% to about 50%, about 3% to about 40%, about 5% to about 30%, about 7% to about 25%, or about 10% to about 20% glycerin. In some embodiments, the toothpaste composition comprises about 0.5%, about 1%, about 3%, about 5%, about 7%, about 9%, about 11%, about 13%, about 15%, about 20%, about 25%, about 30%, about 35%, about 40%, about 45%, or about 50% glycerin. In certain embodiments, the toothpaste composition comprises about 5% glycerin.

In some embodiments, the toothpaste composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w of a thickening agent. In some embodiments, the toothpaste composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w of a thickening agent.

Thickening agents are well known in the art and include without limitation, cellulose gum, xantham gum, maltodextrin, guar gum, carob gum, or acacia gum. In certain embodiments, the thickening agent is cellulose gum.

In some embodiments, the toothpaste composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w cellulose gum. In some embodiments, the toothpaste composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w cellulose gum. In certain embodiments, the toothpaste composition comprises about 1% cellulose gum.

In some embodiments, the toothpaste composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w of a terpene essential oil. In some embodiments, the toothpaste composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w of a terpene essential oil.

In some embodiments, the toothpaste composition of the present disclosure comprises about 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w peppermint oil. In some embodiments, the toothpaste composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w peppermint oil. In certain embodiments, the toothpaste composition comprises about 1% peppermint oil.

In some embodiments, the toothpaste composition of the present disclosure comprises 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w of an emulsifier. In some embodiments, the oral care composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w of an emulsifier.

In some embodiments, the toothpaste composition of the present disclosure comprises 0.001% to about 3%, about 0.005% to about 2.5%, about 0.01% to about 2%, about 0.025% to about 1.5%, about 0.05% to about 1%, about 0.075% to about 0.5%, about 0.1% to about 0.4%, or about 0.2% to about 0.3% w/w quillaja/yucca saponin. In some embodiments, the oral care composition comprises about 0.001%, about 0.005%, about 0.01%, about 0.05%, about 0.1%, about 0.2%, about 0.3%, about 0.4%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.3%, about 1.4%, about 1.5%, about 1.8%, about 2.0%, about 2.3%, about 2.6%, about 2.8%, about 3.0%, about 3.3%, about 3.6%, about 3.8%, about 4.0%, about 4.3%, about 4.6%, about 4.8%, or about 5% w/w quillaja/yucca saponin. In certain embodiments, the toothpaste composition comprises about 1% quillaja/yucca saponin.

In some embodiments, the toothpaste composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w CoQ10. In some embodiments, the toothpaste composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w CoQ10.

In some embodiments, the toothpaste composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w annatto seed extract. In some embodiments, the toothpaste composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w annatto seed extract.

In some embodiments, the toothpaste composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w colloidal silver. In some embodiments, the toothpaste composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w colloidal silver.

In some embodiments, the toothpaste composition of the present disclosure comprises about 0.001% to about 5%, about 0.0025% to about 4%, 0.005% to about 3%, about 0.0075% to about 2%, about 0.01% to about 1.5%, about 0.05% to about 1%, about 0.075% to about 0.75%, about 0.1% to about 0.5% w/w Lactobacillus paracasei. In some embodiments, the toothpaste composition comprises about 0.001%, about 0.0025%, about 0.005%, about 0.0075%, about 0.01%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.8%, about 0.9%, about 1%, about 1.25%, about 1.5%, about 1.75%, about 2%, about 2.5%, about 3%, about 3.5%, about 4%, about 4.5%, or about 5% w/w Lactobacillus paracasei.

In some embodiments, the toothpaste composition of the present disclosure is substantially free of sodium dodecyl sulfate (SDS) or sodium lauryl sulfate (SLS).

Further provided herein are dental devices or oral care kits compatible for human administration which address the unmet needs described above. In exemplary aspects, the dental device is a dental floss, a tongue scraper, a dental pick, a mouth guard, or an orthodontic corrective device.

Further provided herein is a disposable toothbrush for use in oral hygiene. In exemplary aspects, the disposable toothbrush comprises an abrasive cleaning surface or bristles impregnated with any of the compositions disclosed herein. In some embodiments, the abrasive cleaning surface or bristles are impregnated with a toothpaste composition disclosed herein. In some embodiments, the abrasive cleaning surface or bristles are impregnated with a toothpaste composition comprising about 1% to about 5% w/w GG, about 30% to about 40% xylitol, about 1% to about 20% calcium phosphate, about 20% to about 30% w/w glycerin, about 1% to about 2% w/w peppermint oil, about 0.1% to about 2% w/w cellulose gum, about 1% to about 2% w/w quillaja/yucca saponin, and about 5% to about 10% w/w water. In some embodiments, the toothpaste composition comprises: about 1.0% to about 5.0% w/w geranylgeraniol, about 30% to about 40% w/w xylitol, about 10% to about 15% w/w calcium phosphate, about 20% to about 30% w/w glycerin, about 1% to about 2% w/w peppermint oils, about 1% to about 2% w/w cellulose gum, about 1% to about 2% w/w quillaja saponin or yucca saponin, and about 15% to about 25% w/w water. In some embodiments, the abrasive cleaning surface or bristles are impregnated with a toothpaste composition which is substantially free of sodium dodecyl sulfate (SDS) or sodium lauryl sulfate (SLS). As used herein, the term “impregnated” refers to the composition being associated with, contained in the surface, such as an abrasive cleaning surface or bristles, which can be part of a toothbrush or teeth cleaning implement or apparatus. Further provided herein are dental products compatible for human administration which address the unmet needs described above. In exemplary aspects, the dental product is a dental cement, a bone cement, a dental bonding agent, a dental porcelain powder, a pulp capping material, a retrograde filling material, a root canal filling material, or a composite resin filling material, comprising any of the compositions disclosed herein. In some embodiments, the dental product is a dental cement. In some embodiments, the dental cement comprises GG and calcium phosphate.

### Methods of Use and Manufacture

Further provided herein are methods of: (a) promoting gum growth, renewal, wound healing, or tissue repair; (b) removing gum adhesives; (c) preventing gum decay; (d) maintaining gum energetics; (c) improving oral health; (f) reducing plaque accumulation; (g) reducing or inhibiting gingivitis; (h) inhibiting or disrupting microbial biofilm formation in the oral cavity; (i) reducing or inhibiting formation of dental caries; (j) reducing levels of periodontopathogens; (k) reducing levels of anaerobes; (l) reducing levels of acid producing bacteria; (m) increasing relative levels of arginolytic bacteria; (n) immunizing the teeth against cariogenic bacteria; (o) reducing levels of lactic acid; (p) promoting salivary production; (q) treating, relieving, or reducing dry mouth; (r) reducing, repairing, or inhibiting early enamel lesions; (s) promoting dentin regeneration; (t) reducing or inhibiting demineralization or promoting remineralization of the teeth; (u) reducing hypersensitivity of the teeth; (v) reducing periodontal pocket depth; (w) reducing or treating bleeding on dental probing; (x) reducing or inhibiting clinical attachment loss and improving clinical attachment; (y) reducing halitosis; (z) reducing tooth staining; (aa) enhancing salivary CoQ₁₀; (bb) promoting healing of sores or cuts in the mouth or promoting wound healing of gum or enamel; (cc) cleaning the teeth and oral cavity; (dd) promoting systemic health, including cardiovascular health; (ee) preventing or restoring buccal root death or gum death; or (ff) reducing severity of gingival inflammation, comprising applying any of the compositions disclosed herein to the gum, tooth, tooth tissue, enamel, oral cavity, or tooth socket, or any combination thereof, comprising applying any of the oral care compositions described herein to the gum, tooth, tooth tissue, enamel, oral cavity, or tooth socket in a patient or subject in need thereof.

In some embodiments, the periodontopathogen in (j) is a Streptococcus, a Porphyromonas, and Actinomyces, a Lactobacillus, a Spirochaete, a Treponema, an Aggregatibacter, an Enteroccocus, a Candida, or any combination thereof. In some embodiments, the Streptococcus is Streptococcus mutans, Streptococcus sanguinis, Streptococcus goronii, or Streptococcus sobrinus. In some embodiments, the Treponema is Treponema denticola. In some embodiments, the Porphyromonas is Porphyromonas gingivalis. In some embodiments, the Aggregatibacter is Aggregatibacter actinomycetemcomitans. In some embodiments, the Enterococcus is Enterococcus faecalis. In some embodiments, the Candida is Candida albicans.

Further provided herein are methods of: (a) preventing or treating a jaw-bone related syndrome or disease; (b) promoting wound healing of the gum or enamel; (c) restoring the root canal; (d) preventing or treating dental caries, root caries, tooth fracture, root fracture, cervical abrasion, tooth wearing, or gum or enamel loss; or (e) preventing or treating dry socket (alveolar osteitis), comprising applying a dental product disclosed herein to the gum, tooth tissue, enamel, oral cavity, or tooth socket of a patient or subject in need thereof.

Further provided herein are processes of making any of the oral care compositions disclosed herein. In some embodiments, the process of making the oral care composition comprises mixing GG and the orally acceptable carrier.

### Examples

The following examples are provided to further describe some of the embodiments disclosed herein. The examples are intended to illustrate, not to limit, the disclosed embodiments.

### Example 1. Oil-Based Oral Care Composition A

| Ingredient | Amount (per ~15-20 ml serving) | |
|---|---|---|
| Medium chain triglycerides | q.s. | |
| CoQ10 | 100-200 | mg |
| Geranylgeraniol | 40 | mg |
| Bromelain | 80-500 | mg |
| Cinnamon | 0.05-0.15 | mL |

*q.s. meaning an amount that makes the total amount to 15-20 ml.

### Example 2. Oil-Based Oral Care Composition B

| Ingredient | Amount (per ~15 ml serving) | |
|---|---|---|
| GG Gold ® 75 Oil* | 141.00 | mg |
| DeltaGold ® 70 Oil** | 7.05 | mg |
| Bromelain 2400GDU*** | 70.50 | mg |
| CoQ10 (Ubiquinone) | 7.05 | mg |
| Natural Mint Flavor WONF FASX064 | 112.80 | mg |
| LCO 100 ® MCT Oil**** | 13.76 | g |

*provided by American River Nutrition. GG Gold ® 75 oil contains a minimum of 75% w/w pure GG.
**provided by American River Nutrition. DeltaGold ® 70 oil contains a minimum of 70% w/w pure tocotrienols, comprising 84-92% w/w delta-tocotrienol and 8-16% w/w gamma-tocotrienol.
***provided by Maypro Industries. Bromelain 2400 GDU (Gelatin Digesting Units) has an activity of 2400 GDU/g.
****provided by Bioriginal. LCO 100 ® MCT oil contains a minimum of 35% w/w caprylic acid, 25% w/w capric acid, and 26% w/w lauric acid.

### Example 3. Oil-Based Oral Care Composition C

| Ingredient | Amount (per ~15 ml serving) | |
|---|---|---|
| GG Gold ® 75 Oil* | 141.00 | mg |
| DeltaGold ® 70 Oil** | 7.05 | mg |
| Bromelain 2400GDU*** | 70.50 | mg |
| CoQ10 (Ubiquinone) | 7.05 | mg |
| Natural Lemon Flavor WONF FASX062 | 141.00 | mg |
| LCO 100 ® MCT Oil**** | 13.73 | g |

*provided by American River Nutrition. GG Gold ® 75 oil contains a minimum of 75% w/w pure GG.
**provided by American River Nutrition. DeltaGold ® 70 oil contains a minimum of 70% w/w pure tocotrienols, comprising 84-92% w/w delta-tocotrienol and 8-16% w/w gamma-tocotrienol.
***provided by Maypro Industries. Bromelain 2400 GDU (Gelatin Digesting Units) has an activity of 2400 GDU/g.
****provided by Bioriginal. LCO 100 ® MCT oil contains a minimum of 35% w/w caprylic acid, 25% w/w capric acid, and 26% w/w lauric acid.

### Example 4. Oil-Based Toothpaste Composition A

| | Amount in the |
|---|---|
| Ingredient | composition |
| Xylitol | 5-40% (~35%) |
| Calcium phosphate | 0-40% (~30%) |
| Purified water | 5-30% (~25%) |
| Glycerin | 0-10% (~5%) |
| GG | 0.5-5% (~1%) |
| Menthol and Peppermint Oil | 0.5-2% (~1%) |
| Cellulose Gum | 0.5-2% (~1%) |
| Quillaja/Yucca Saponin | 0.5-2% (~1%) |

### Example 5. Oil-Based Toothpaste Composition B

| | Amount in the |
|---|---|
| Ingredient | composition |
| Xylitol | 30-40% |
| Calcium phosphate | 10-15% |
| Purified water | 15-25% |
| Glycerin | 20-30% |
| GG | 1-5% |
| Menthol and Peppermint Oil | 1-2% |
| Cellulose Gum | 1-2% |
| Quillaja/Yucca Saponin | 1-2% |

### Example 6. Compositions and Preparation in Topical/Local Therapeutic and Dental Bone Cement

GG is applied as an oil in an amount ranging from 60-95% (˜75%) dependent on the topical protein gel or dental cement wherein the powder-matrices are physically mixed. The final GG concentration is 0.05-0.1% in a specific matrix or cement, however, compositions wherein the final GG concentration range is 1-5% may also be prepared.

### Example 7. Oil Pull Composition Method of Use

A subject uses the oil pull composition in the morning after normal brushing that caused mild abrasion. An approximate 15 mL (one tablespoon) of the composition was actively swished in the mouth for 1-10 min (˜5 min). Such application provides GG and xylitol gum contact and enamel contact. This soothes the gums and reduces oral acid. Such applications are applied about 2-3 times per day, especially after each tooth brushing activity.

### Example 8. Preparation of Toothbrush Impregnated with Toothpaste Comprising GG

First, the impregnated toothpaste is water-hydrated to emulsify the oily GG. Second, the paste is dried, and re-adhered to simulate impregnation to bristles. Third, GG is incorporated into the toothpaste without paste separation nor paste flaking. GG is emulsified 5-20%, above the expected 1-5% GG.

### Example 9. Method of Use for Toothbrush Impregnated with Toothpaste Comprising GG

A subject uses a toothbrush with toothpaste comprising GG for 1-2 min. It freshens the breath without an off-flavor taste. The subject repeats tooth brushing about 2 hours later after a meal. Besides experiencing freshened breath, the patient's oral cavity also feels less acidic.

### Example 10. Ingredient List of Toothpaste Comprising GG

Geranylgeraniol, Glycerin, Water, Hydrated Silica, Calcium Carbonate, Coco-Betaine, Cellulose Gum, Xylitol, Xanthan Gum, Flavor, Rebaudioside A, Quillaja or Yucca Saponaria Extract, Sodium Bicarbonate, Maltodextrin, Ubiquinone, Lactobacillus Paracasei, Vitis Vinifera (Grape) Seed Extract, Bixa Orellana Seed Extract, and Colloidal Silver.

### Example 11. Clinical Study

### I. Criteria for Subject Selection

Number of Participants: 18 participants.

Gender of Participants: Male and female participants will be eligible to participate.

Age of Participants: Adults 18 years of age and older will be eligible to participate.

Racial and Ethnic Origin: Participants of all races and ethnicities are eligible to participate in this study.

Inclusion Criteria: The study inclusion criteria are explicitly defined as follows:
1. Age≥18 years
2. Plaque index of at least 1
3. Gingival index of at least 1
4. Pocket depth no greater than 5 mm
5. Able to understand and write English
6. Voluntarily consent to the study and understand its nature and purpose including potential risks and side effects
7. Maintain current dental hygiene routine
8. Bleeding on probing

Exclusion Criteria: The study exclusion criteria were formulated on the basis of scientific and clinical input and are defined as follows:
1. Current daily use of any products containing the nutrients and/or herbs in the study product
2. Known allergies to any substance in the study product
3. Current daily tobacco smoker
4. Currently pregnant or lactating women or women planning to become pregnant in the next 12 weeks
5. Active oral infection (ie. herpes, candida)
6. Recently on antibiotics (past 3 months)
7. Undergone recent periodontal therapy (last 6 months)

Vulnerable Participants: No children, pregnant women, nursing home residents or other institutionalized persons, prisoners, or any other vulnerable participants will be eligible to participate in this study.

### II. Methods and Procedures

Study Design Overview: The proposed study is a 12-week pilot study on oil-based oral care compositions B and C. Eligible participants will be recruited from within the clinical practice of the periodontist. All study outcomes will be measurements and testing during three dental visits spaced 6 weeks apart.

Intervention: Participants will swish 1 teaspoon (approx. 5 mL) of oil in the mouth for 1-3 minutes after their normal brushing routine. They will spit out the oil and not rinse to allow for longer contact to the teeth and gums. This will be once a day for 12 weeks.

Study Population and Inference: As reflected in the study inclusion/exclusion criteria, the study population will consist exclusively of adults with measurements reflecting a specific state of dental health.

### III. Study Outcomes

Study outcomes will consist of parameters of dental health among a sample of adults. These outcomes will be collected during a dental visit and assessed at baseline, six weeks, and at the conclusion of the 12-week clinical trial.

Oral samples that are obtained during clinical visits will be sent to lab utilized by participating clinical practices for analysis of study outcomes.

The outcomes in this clinical trial have been studied in previous clinical trials among dental patients. One oral sample at each of the three study visits is all that is required to collect outcomes. The specific outcomes and measurements assessed at baseline, six weeks, and at the 12-week follow up visit are as follows:

Plaque Index—Primary Outcome—assesses the amount of dental plaque visible on the vestibular and lingual surfaces of all teeth, except the third molars. The bacterial plaque developer solution was used to define cumulative amounts of plaque with criteria from 0 to 5. Once the values of the individual teeth are recorded, they are added and divided by the number of teeth examined to obtain the plaque index of each patient.

Gingival Index—Primary Outcome—a method of recording the clinical severity of gingival inflammation. It is based upon probe measurement of periodontal pockets and on gingival tissue status.

Pocket Depth—Primary Outcome—a measurement in millimeters of the depth from the gingival margin to the epithelial attachment in unhealthy gingival tissue. The pocket depth usually consists of depths great than 3 mm. pocket.

Intraoral Photos—Secondary Outcome—photos taken of the teeth, gums, and oral tissue. These may be a single tooth, a group of teeth, or any area of the mouth. Intraoral photos help establish a baseline for all dental conditions and are used as a tool to monitor any recession or suspicious lesions.

MyPerioPath®—Secondary Outcome—used test for the detection of oral pathogens that cause gum disease and threaten oral & systemic health. It provides early warning of oral pathogens to enable the personalization of periodontal treatment. This test is non-invasive oral rinse collection (OralDNA® Labs).

### Example 12. Study of GG Antimicrobial Properties

The GG Gold® 75 Oil was provided by American River Nutrition. GG Gold® 75 oil contains a minimum of 75% w/w pure GG. GG's antimicrobial properties were tested using GG Gold® 75 Oil on Streptococcus mutan, which is the bacterium most commonly associated with plaque formation and tooth decay.

Tests were performed to obtain Minimum Inhibitory Concentration (MIC) and Minimum Bactericidal Concentration (MBC) described below. The results showed that GG can effectively inhibit growth of oral bacteria.

Minimum Inhibitory Concentration (MIC) is the lowest concentration of the GG Gold® 75 Oil needed to prevent visible growth of the bacterium. The test was accomplished through a broth dilution assay with serial dilutions into multiple tubes to obtain a gradient for the test compound, whereas microbial inoculations were the same in each test tube. The MIC was found to be 9.53%. The MIC was measured under at least 75% potency of pure GG since the GG Gold® 75 oil contains a minimum of 75% w/w pure GG.

Minimum Bactericidal Concentration (MBC) is the lowest concentration of the GG Gold® 75 Oil required to kill a bacterium. The test was accomplished by plating onto agar plates to count colony-forming units. The MBC was found to be 47.7%. MBC was measured under at least 75% potency of pure GG since the GG Gold® 75 oil contains a minimum of 75% w/w pure GG.

### REFERENCES

1. Aas, J. A., et al., Defining the normal bacterial flora of the oral cavity. J Clin Microbiol, 2005. 43(11): p. 5721-32.
2. Kulik, E. M., et al., Identification of archaeal rDNA from subgingival dental plaque by PCR amplification and sequence analysis. FEMS Microbiol Lett, 2001. 196(2): p. 129-33.
3. Keijser, B. J., et al., Pyrosequencing analysis of the oral microflora of healthy adults. J Dent Res, 2008. 87(11): p. 1016-20.
4. Kreth, J., J. Merritt, and F. Qi, Bacterial and host interactions of oral streptococci. DNA Cell Biol, 2009. 28(8): p. 397-403.
5. Huang, R., M. Li, and R. L. Gregory, Bacterial interactions in dental biofilm. Virulence, 2011. 2(5): p. 435-44.
6. Perez-Chaparro, P. J., et al., Newly identified pathogens associated with periodontitis: a systematic review. J Dent Res, 2014. 93(9): p. 846-58.
7. Henderson, B., J. M. Ward, and D. Ready, Aggregatibacter (Actinobacillus) actinomycetemcomitans: a triple A* periodontopathogen? Periodontol 2000, 2010. 54(1): p. 78-105.
8. Alghamdi, F. and M. Shakir, The Influence of Enterococcus faecalis as a Dental Root Canal Pathogen on Endodontic Treatment: A Systematic Review. Cureus, 2020. 12(3): p. e7257.
9. Du, Q., et al., Candida albicans promotes tooth decay by inducing oral microbial dysbiosis. ISME J, 2021. 15(3): p. 894-908.
10. Carter, W. J., et al., The formation of lactic acid in dental plaques. I. Caries-active individuals. J Dent Res, 1956. 35(5): p. 778-85.
11. Naseem, M., et al., Oil pulling and importance of traditional medicine in oral health maintenance. Int J Health Sci (Qassim), 2017. 11(4): p. 65-70.
12. Tomar, P., et al., Oil pulling and oral health: A review. Int J Sci Study, 2014. 1: p. 33.
13. Kabara, J. J., et al., Fatty acids and derivatives as antimicrobial agents. Antimicrob Agents Chemother, 1972. 2(1): p. 23-8.
14. Huang, C. B., et al., Short- and medium-chain fatty acids exhibit antimicrobial activity for oral microorganisms. Arch Oral Biol, 2011. 56(7): p. 650-654.
15. Ripari, F., et al., The Role of Coconut Oil in Treating Patients Affected by Plaque-Induced Gingivitis: A Pilot Study. Eur J Dent, 2020. 14(4): p. 558-565.
16. Sezgin, Y., et al., Comparison of the plaque regrowth inhibition effects of oil pulling therapy with sesame oil or coconut oil using 4-day plaque regrowth study model: A randomized crossover clinical trial. Int J Dent Hyg, 2021.
17. Naseem, M., et al., Oil pulling and importance of traditional medicine in oral health maintenance. Int J Health Sci (Qassim), 2017. 11(4): p. 65-70.
18. Tomar, P., et al., Oil pulling and oral health: A review. Int J Sci Study, 2014. 1: p. 33.
19. Kabara, J. J., et al., Fatty acids and derivatives as antimicrobial agents. Antimicrob Agents Chemother, 1972. 2(1): p. 23-8.
20. Huang, C. B., et al., Short- and medium-chain fatty acids exhibit antimicrobial activity for oral microorganisms. Arch Oral Biol, 2011. 56(7): p. 650-654.
21. Ripari, F., et al., The Role of Coconut Oil in Treating Patients Affected by Plaque-Induced Gingivitis: A Pilot Study. Eur J Dent, 2020. 14(4): p. 558-565.
22. Sezgin, Y., et al., Comparison of the plaque regrowth inhibition effects of oil pulling therapy with sesame oil or coconut oil using 4-day plaque regrowth study model: A randomized crossover clinical trial. Int J Dent Hyg, 2021.
23. Asokan, S., P. Emmadi, and R. Chamundeswari, Effect of oil pulling on plaque induced gingivitis: a randomized, controlled, triple-blind study. Indian J Dent Res, 2009. 20(1): p. 47-51.
24. Asokan, S., et al., Effect of oil pulling on halitosis and microorganisms causing halitosis: a randomized controlled pilot trial. J Indian Soc Pedod Prev Dent, 2011. 29(2): p. 90-4.
25. Asokan, S., et al., Effect of oil pulling on Streptococcus mutans count in plaque and saliva using Dentocult SM Strip mutans test: a randomized, controlled, triple-blind study. J Indian Soc Pedod Prev Dent, 2008. 26(1): p. 12-7.
26. Kaushik, M., et al., The Effect of Coconut Oil pulling on Streptococcus mutans Count in Saliva in Comparison with Chlorhexidine Mouthwash. J Contemp Dent Pract, 2016. 17(1): p. 38-41.
27. Peedikayil, F. C., et al., Comparison of antibacterial efficacy of coconut oil and chlorhexidine on Streptococcus mutans: An in vivo study. J Int Soc Prev Community Dent, 2016. 6(5): p. 447-452.
28. Sezgin, Y., B. M. Ozgul, and N. O. Alptekin, Efficacy of oil pulling therapy with coconut oil on four-day supragingival plaque growth: A randomized crossover clinical trial. Complement Ther Med, 2019. 47: p. 102193.
29. Sood, P., et al., Comparative efficacy of oil pulling and chlorhexidine on oral malodor: a randomized controlled trial. J Clin Diagn Res, 2014. 8(11): p. ZC18-21.
30. Porter, S. R. and C. Scully, Oral malodour (halitosis). BMJ, 2006. 333(7569): p. 632-635.
31. Festenstein, G. N., et al., A constituent of the unsaponifiable portion of animal tissue lipids (lambda max. 272 m mu). Biochem J, 1955. 59(4): p. 558-66.
32. Olson, R. E. and H. Rudney, Biosynthesis of ubiquinone. Vitam Horm, 1983. 40: p. 1-43.
33. Crane, F. L., et al., Isolation of a quinone from beef heart mitochondria. Biochim Biophys Acta, 1957. 25(1): p. 220-1.
34. Ernster, L. and G. Dallner, Biochemical, physiological and medical aspects of ubiquinone function. Biochim Biophys Acta, 1995. 1271(1): p. 195-204.
35. Kalen, A., E. L. Appelkvist, and G. Dallner, Age-related changes in the lipid compositions of rat and human tissues. Lipids, 1989. 24(7): p. 579-84.
36. Zozina, V. I., et al., Coenzyme Q10 in Cardiovascular and Metabolic Diseases: Current State of the Problem. Curr Cardiol Rev, 2018. 14(3): p. 164-174.
37. Ferlazzo, N., et al., Changes in the Biomarkers of Oxidative/Nitrosative Stress and Endothelial Dysfunction Are Associated with Cardiovascular Risk in Periodontitis Patients. Curr Issues Mol Biol, 2021. 43(2): p. 704-715.
38. Bullon, P., et al., Mitochondrial dysfunction promoted by Porphyromonas gingivalis lipopolysaccharide as a possible link between cardiovascular disease and periodontitis. Free Radic Biol Med, 2011. 50(10): p. 1336-43.
39. Nakamura, R., et al., Study of CoQ10-enzymes in gingiva from patients with periodontal disease and evidence for a deficiency of coenzyme Q10. Proc Natl Acad Sci USA, 1974. 71(4): p. 1456-60.
40. Wolf, D. E., Hoffmann, C. H., Trenner, N. R., Anson, D. H., Shunk, C. H., Linn, B. O., McPherson, J. F., Folkers, K., Coenzyme Q. Structural studies coenzyme Q group. Am Chem Soc, 1958. 80: p. 4752.
41. Wilkinson, E. G., et al., Bioenergetics in clinical medicine. Il. Adjunctive treatment with coenzyme Q in periodontal therapy. Res Commun Chem Pathol Pharmacol, 1975. 12(1): p. 111-23.
42. Prakash, S., J. Sunitha, and M. Hans, Role of coenzyme Q(10) as an antioxidant and bioenergizer in periodontal diseases. Indian J Pharmacol, 2010. 42(6): p. 334-7.
43. Hanioka, T., et al., Effect of topical application of coenzyme Q10 on adult periodontitis. Mol Aspects Med, 1994. 15 Suppl: p. s241-8.
44. Hans, M., S. Prakash, and S. Gupta, Clinical evaluation of topical application of perio-Q gel (Coenzyme Q(10)) in chronic periodontitis patients. J Indian Soc Periodontol, 2012. 16(2): p. 193-9.
45. Pranam, S., et al., Evaluation of Efficacy of Coenzyme Q10 as an Adjunct to Nonsurgical Periodontal Therapy and Its Effect on Crevicular Superoxide Dismutase in Patients with Chronic Periodontitis. Eur J Dent, 2020. 14(4): p. 551-557.
46. Sale, S. T., et al., A comparative evaluation of topical and intrasulcular application of coenzyme Q10 (Perio Q) gel in chronic periodontitis patients: A clinical study. J Indian Soc Periodontol, 2014. 18(4): p. 461-5.
47. Manthena, S., et al., Effectiveness of CoQ10 Oral Supplements as an Adjunct to Scaling and Root Planing in Improving Periodontal Health. J Clin Diagn Res, 2015. 9(8): p. ZC26-8.
48. Chatterjee, A., et al., Evaluation of Co-Q10 anti-gingivitis effect on plaque induced gingivitis: A randomized controlled clinical trial. J Indian Soc Periodontol, 2012. 16(4): p. 539-42.
49. Shaheen, M. A., et al., Innovative coenzyme Q10-loaded nanoformulation as an adjunct approach for the management of moderate periodontitis: preparation, evaluation, and clinical study. Drug Deliv Transl Res, 2020. 10(2): p. 548-564.
50. Raut, C. P., et al., Subgingivally delivered coenzyme Q10 in the treatment of chronic periodontitis among smokers: A randomized, controlled clinical study. J Oral Biol Craniofac Res, 2019. 9(2): p. 204-208.
51. Matthews-Brzozowska, T., Kurhanska-Flisykowska, A., Stop, J., Healing of periodontal tissue assisted by Coenzyme Q10 with Vitamin E—clinical and laboratory evaluation. Pharmacological Reports, 2007. 59.
52. Raut, C. P. and K. S. Sethi, Comparative evaluation of co-enzyme Q10 and Melaleuca alternifolia as antioxidant gels in treatment of chronic periodontitis: A clinical study. Contemp Clin Dent, 2016. 7(3): p. 377-81.
53. Sharma, V., et al., Comparative evaluation of coenzyme Q10-based gel and 0.8% hyaluronic acid gel in treatment of chronic periodontitis. J Indian Soc Periodontol, 2016. 20(4): p. 374-380.
54. Ryo, K., et al., Effects of coenzyme Q10 on salivary secretion. Clin Biochem, 2011. 44(8-9): p. 669-74.
55. Ushikoshi-Nakayama, R., et al., Effect of gummy candy containing ubiquinol on secretion of saliva: A randomized, double-blind, placebo-controlled parallel-group comparative study and an in vitro study. PLOS One, 2019. 14(4): p. e0214495.
56. Yoneda, T., et al., Application of coenzyme Q10 for accelerating soft tissue wound healing after tooth extraction in rats. Nutrients, 2014. 6(12): p. 5756-69.
57. Fedeli, E., et al., Isolation of geranyl geraniol from the unsaponifiable fraction of linseed oil. J Lipid Res, 1966. 7(3): p. 437-41.
58. Lange, B. M., et al., Isoprenoid biosynthesis: the evolution of two ancient and distinct pathways across genomes. Proc Natl Acad Sci USA, 2000. 97(24): p. 13172-7.
59. Wagner-Jauregg, T., Synthesen von Terpenen aus Isopren. Ann Chem, 1932. 52(496): p. 52-77.
60. Crick, D. C., C. J. Waechter, and D. A. Andres, Utilization of geranylgeraniol for protein isoprenylation in C6 glial cells. Biochem Biophys Res Commun, 1994. 205(1): p. 955-61.
61. Ho, H. J., et al., A novel function of geranylgeraniol in regulating testosterone production. Biosci Biotechnol Biochem, 2018. 82(6): p. 956-962.
62. Stoffel, W. and C. Martius, Zur Synthese der K-Vitamine und Ubichinone. Angew Chem, 1960. 72(17): p. 627-627.
63. McCormack, M. G., et al., Staphylococcus aureus and the oral cavity: an overlooked source of carriage and infection? Am J Infect Control, 2015. 43(1): p. 35-7.
64. Inoue, Y., et al., Biphasic effects of geranylgeraniol, teprenone, and phytol on the growth of Staphylococcus aureus. Antimicrob Agents Chemother, 2005. 49(5): p. 1770-4.
65. Sharma, S., et al., Oral tuberculosis—Current concepts. J Family Med Prim Care, 2019. 8(4): p. 1308-1312.
66. Vik, A., A. James, and L. L. Gundersen, Screening of terpenes and derivatives for antimycobacterial activity; identification of geranylgeraniol and geranylgeranyl acetate as potent inhibitors of Mycobacterium tuberculosis in vitro. Planta Med, 2007. 73(13): p. 1410-2.
67. Lopes, M. V., et al., Mitochondria Superoxide Anion Production Contributes to Geranylgeraniol-Induced Death in Leishmania amazonensis. Evid Based Complement Alternat Med, 2012. 2012: p. 298320.
68. Unoshima, M., et al., Antiviral effects of geranylgeranylacetone: enhancement of MxA expression and phosphorylation of PKR during influenza virus infection. Antimicrob Agents Chemother, 2003. 47(9): p. 2914-21.
69. Reszka, A. A. and G. A. Rodan, Nitrogen-containing bisphosphonate mechanism of action. Mini Rev Med Chem, 2004. 4(7): p. 711-9.
70. Gupta, M. and N. Gupta, Bisphosphonate Related Jaw Osteonecrosis, in StatPearls. 2022: Treasure Island (FL).
71. Koneski, F., et al., Geranylgeraniol Reverses the Toxicity Induced by Clinical Doses of Zoledronic Acid on Gingival Epithelial Cells and Gingival Fibroblasts. Dental Sciences Maxillofacial Surgery, 2021. 9.
72. Cozin, M., et al., Novel therapy to reverse the cellular effects of bisphosphonates on primary human oral fibroblasts. J Oral Maxillofac Surg, 2011. 69(10): p. 2564-78.
73. Neel, E. A. A., et al., Demineralization-remineralization dynamics in teeth and bone. Int J Nanomedicine, 2016. 11: p. 4743-4763.
74. Mungpayabarn, H. and S. Patntirapong, Timing of geranylgeraniol addition increases osteoblast activities under alendronate condition. J Oral Biol Craniofac Res, 2021. 11(3): p. 396-401.
75. Singhatanadgit, W., W. Hankamolsiri, and W. Janvikul, Geranylgeraniol prevents zoledronic acid-mediated reduction of viable mesenchymal stem cells via induction of Rho-dependent YAP activation. R Soc Open Sci, 2021. 8(6): p. 202066.
76. Zafar, S., et al., Effects of zoledronic acid and geranylgeraniol on angiogenic gene expression in primary human osteoclasts. J Oral Sci, 2020. 62(1): p. 79-83.
77. Hiruma, Y., et al., Vitamin K2 and geranylgeraniol, its side chain component, inhibited osteoclast formation in a different manner. Biochem Biophys Res Commun, 2004. 314(1): p. 24-30.
78. Politis, C., et al., Wound Healing Problems in the Mouth. Front Physiol, 2016. 7: p. 507.
79. Chen, X., et al., Geranylgeraniol Restores Zoledronic Acid-Induced Efferocytosis Inhibition in Bisphosphonate-Related Osteonecrosis of the Jaw. Front Cell Dev Biol, 2021. 9: p. 770899.
80. Koneski, F., et al., In vivo effects of geranylgeraniol on the development of bisphosphonate-related osteonecrosis of the jaws. J Craniomaxillofac Surg, 2018. 46(2): p. 230-236.
81. Ghensi, P., et al., In Vitro Effect of Bromelain on the Regenerative Properties of Mesenchymal Stem Cells. J Craniofac Surg, 2019. 30(4): p. 1064-1067.
82. Inchingolo, F., et al., Clinical trial with bromelain in third molar exodontia. Eur Rev Med Pharmacol Sci, 2010. 14(9): p. 771-4.
83. Kumar, S. T., et al., Postoperative Healing after Surgical Removal of Mandibular Third Molar: A Comparative Study between Two Proteolytic Enzymes. J Pharm Bioallied Sci, 2020. 12: p. S289-S294.
84. Bormann, K. H., et al., Perioperative Bromelain Therapy after Wisdom Teeth Extraction—A Randomized, Placebo-Controlled, Double-Blinded, Three-Armed, Cross-Over Dose-Finding Study. Phytother Res, 2016. 30(12): p. 2012-2019.
85. Singh, T., et al., Effect of proteolytic enzyme bromelain on pain and swelling after removal of third molars. J Int Soc Prev Community Dent, 2016. 6(Suppl 3): p. S197-S204.
86. Soheilifar, S., et al., Effect of Oral Bromelain on Wound Healing, Pain, and Bleeding at Donor Site Following Free Gingival Grafting: A Clinical Trial. J Dent (Tehran), 2018. 15(5): p. 309-316.
87. Hong, J. H., et al., Anti-Inflammatory and Mineralization Effects of Bromelain on Lipopolysaccharide-Induced Inflammation of Human Dental Pulp Cells. Medicina (Kaunas), 2021. 57(6).
88. Tete, G., F. Cattoni, and E. Polizzi, Anti-discoloration system: a new chlorhexidine mouthwash. J Biol Regul Homeost Agents, 2021. 35(4 Suppl. 1): p. 113-118.
89. Kalyana, P., et al., Stain removal efficacy of a novel dentifrice containing papain and Bromelain extracts—an in vitro study. Int J Dent Hyg, 2011. 9(3): p. 229-33.
90. Munchow, E. A., et al., Stain removal effect of novel papain—and bromelain-containing gels applied to enamel. Clin Oral Investig, 2016. 20(8): p. 2315-2320.
91. Chakravarthy, P. and S. Acharya, Efficacy of extrinsic stain removal by novel dentifrice containing papain and bromelain extracts. J Young Pharm, 2012. 4(4): p. 245-9.
92. Patil, P. A., et al., Comparison of effectiveness of abrasive and enzymatic action of whitening toothpastes in removal of extrinsic stains—a clinical trial. Int J Dent Hyg, 2015. 13(1): p. 25-9.
93. Khan, R., et al., Comparison of Different Dentin Deproteinizing Agents on the Shear Bond Strength of Resin-bonded Dentin. Int J Clin Pediatr Dent, 2020. 13(Suppl 1): p. S69-S77.
94. Khatib, M. S., et al., Microtensile Bond Strength of Composite Resin Following the Use of Bromelain and Papain as Deproteinizing Agents on Etched Dentin: An In Vitro Study. Int J Clin Pediatr Dent, 2020. 13(1): p. 43-47.
95. Mulgaonkar, A., et al., Effect of bromelain enzyme on the microleakage of composite resin restorations after external tooth bleaching: An in vitro study. J Conserv Dent, 2019. 22(5): p. 436-440.
96. Balakrishnan, M., R. S. Simmonds, and J. R. Tagg, Dental caries is a preventable infectious disease. Aust Dent J, 2000. 45(4): p. 235-45.
97. Reddy, V. K., et al., Bromelain vs Papain Gel for Caries Removal in Primary Teeth. J Contemp Dent Pract, 2019. 20(11): p. 1345-1349.
98. Abinaya, R., et al., Comparing the Efficacy of Three Minimally Invasive Techniques on Demineralized Dentin in Primary Teeth and Evaluating Its Residual Dentin and Microhardness Levels: An In Vitro Study. Int J Clin Pediatr Dent, 2020. 13(6): p. 585-589.
99. Praveen, N.C., et al., In vitro Evaluation of Antibacterial Efficacy of Pineapple Extract (Bromelain) on Periodontal Pathogens. J Int Oral Health, 2014. 6(5): p. 96-8.
100. Tadikonda, A., et al., Anti-plaque and anti-gingivitis effect of Papain, Bromelain, Miswak and Neem containing dentifrice: A randomized controlled trial. J Clin Exp Dent, 2017. 9(5): p. e649-e653.
101. Yanakiev, S., Effects of Cinnamon (Cinnamomum spp.) in Dentistry: A Review. Molecules, 2020. 25(18).
102. Chaudhari, L. K., et al., Antimicrobial activity of commercially available essential oils against Streptococcus mutans. J Contemp Dent Pract, 2012. 13(1): p. 71-4.
103. Gupta, C., et al., Comparative study of cinnamon oil and clove oil on some oral microbiota. Acta Biomed, 2011. 82(3): p. 197-9.
104. Veloso, D. J., et al., Potential antibacterial and anti-halitosis activity of medicinal plants against oral bacteria. Arch Oral Biol, 2020. 110: p. 104585.
105. Richards, D., Effect of essential oil mouthwashes on plaque and gingivitis. Evid Based Dent, 2017. 18(2): p. 39-40.
106. Gupta, D. and A. Jain, Effect of Cinnamon Extract and Chlorhexidine Gluconate (0.2%) on the Clinical Level of Dental Plaque and Gingival Health: A 4-Week, Triple-Blind Randomized Controlled Trial. J Int Acad Periodontol, 2015. 17(3): p. 91-8.
107. Prajapati, M., et al., Antifungal effects of tulsi, garlic, cinnamon and lemongrass in powder and oil form on Candida albicans: An in vitro study. J Oral Maxillofac Pathol, 2021. 25(2): p. 306-312.
108. Marcoux, E., et al., Antimicrobial activities of natural plant compounds against endodontic pathogens and biocompatibility with human gingival fibroblasts. Arch Oral Biol, 2020. 116: p. 104734.
109. Otto, M., et al., Geranyl-geraniol addition affects potency of bisphosphonates-a comparison in vitro promising a therapeutic approach for bisphosphonate-associated osteonecrosis of the jaw and oral wound healing. Oral Maxillofac Surg, 2021. 26: p. 321-332.
110. Hagelauer, N., et al., Bisphosphonates inhibit cell functions of HUVECs, fibroblasts and osteogenic cells via inhibition of protein geranylgeranylation. Clin Oral Invest, 2014. 19(5): p. 1079-1091.
111. Ziebart, T., et al., Geranylgeraniol—A new potential therapeutic approach to bisphosphonate associated osteonecrosis of the jaw. Oral Oncol, 2011. 47: p. 195-201.
112. Marcuzzi, A., et al., Comments on “Geranylgeraniol—a new potential therapeutic approach to bisphosphonate associated osteonecrosis of the jaw” by Ziebart T et al. (2011). Oral Oncol, 2011. 47(5): p. 436-437.
113. Pabst, A. M., et al., Isoprenoid geranylgeraniol: the influence on cell characteristics of endothelial progenitor cells after bisphosphonate therapy in vitro. Clin Oral Invest, 2015. 19: p. 1625-1633.
114. Pabst, A. M., et al., The influence of geranylgeraniol on microvessel sprouting after bisphosphonate substitution in an in viro 3D-angiogenesis assay. Clin Oral Invest, 2016. 23(3): p. 771-778.
115. Feldman, G. J., et al., Geranylgeraniol in bone cement rescues osteoclasts from the toxic effects of pamidronate and restores function. Preprint, 2020.
116. Ikebe, T., Pathophysiology of BRONJ: Drug-related osteoclastic disease of the jaw. Oral Sci Int, 2013. 10: p. 1-8.
117. Joshipura, K. J., et al., Over-the-counter mouthwash use and risk of pre- diabetes/diabetes. Nitric Oxide, 2017. 71: p. 14-20.
118. Joshipura, K., et al., Over-the-counter mouthwash use, nitric oxide and hypertension risk. Blood Pressure, 2020. 29(2): p. 103-112.

The present embodiments are not to be limited in scope by the specific embodiments described herein. Indeed, various modifications of the embodiments, in addition to those described herein, will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1-125. (canceled)

126. An oral composition comprising geranylgeraniol and an orally acceptable carrier.

127. The composition of claim 126, wherein the composition is an oral rinse, a toothpaste, a mouth spray, a tooth powder, or a chewing gum.

128. The composition of claim 126, wherein the composition is an oral rinse product.

129. The composition of claim 128, wherein the orally acceptable carrier is medium chain triglycerides, short chain triglycerides, long chain triglycerides, coconut oil, palm kernel oil, sesame oil, sunflower oil, or any combination thereof.

130. The composition of claim 129, wherein the medium chain triglycerides comprise caprylic acid, capric acid, lauric acid, or any combination thereof, or wherein the short chain triglyceride is glyceryl triacetate (triacetin), glyceryl tripropionate, or glyceryl butyrate.

131. The composition of claim 128, wherein the composition comprises about 0.1% to about 5% w/w geranylgeraniol.

132. The composition of claim 128, wherein:
a) the composition further comprises one or more selected from the group consisting of an antioxidant, a protease, and a flavoring agent; and/or
b) the composition further comprises one of more selected from the group consisting of a terpene essential oil, ascorbyl palmitate, and saponins.

133. The composition of claim 132, wherein:
a) the antioxidant is CoQ₁₀, tocotrienol, menaquinone, or any of the combinations thereof;
b) the protease is bromelain;
c) the terpene essential oil is cinnamon oil, tea tree oil, lemongrass oil, clove oil, peppermint oil, spearmint oil, wintergreen oil, cedarwood oil, lemon oil, lime oil, eucalyptus oil, oregano oil, or any combination thereof; and/or
d) the saponins are quillaja saponins or yucca saponins.

134. The composition of claim 133, wherein:
a) the CoQ₁₀ is ubiquinol, ubiquinone, or a mixture of ubiquinol and ubiquinone;
b) menaquinone comprises MK-4, MK-7, MK-8, MK-9, MK-10, MK-11, MK-12, MK-13, or any combinations thereof;
c) the tocotrienol is derived from annatto, palm, or rice;
d) the composition comprises about 0.01% to about 1.5% w/w CoQ₁₀;
e) the composition comprises about 0.01% to about 1% w/w tocotrienol; and/or
f) the composition comprises about 0.05% to about 5% w/w bromelain.

135. The composition of claim 132, wherein the composition comprises:
i) about 0.01% to about 3% w/w terpene essential oil,
ii) about 0.1% to about 5% w/w ascorbyl palmitate, and/or
iii) about 0.1% to about 5% w/w quillaja saponin or yucca saponin.

136. The composition of claim 128, wherein the composition comprises:
i) about 0.1% to about 5% w/w geranylgeraniol,
ii) about 0.01% to about 1% w/w CoQ₁₀,
iii) about 0.01% to about 1% w/w tocotrienol,
iv) about 0.05% to about 5% w/w bromelain, and
v) about 0.01% to about 3% w/w terpene essential oil.

137. The composition of claim 128, wherein:
a) the weight ratio of geranylgeraniol to the orally acceptable carrier ranges from about 1:3000 to about 1:40;
b) the weight ratio of geranylgeraniol to CoQ₁₀ ranges from about 1:1 to about 20:1;
c) the weight ratio of geranylgeraniol to tocotrienol ranges from about 1.5:1 to about 5:1 or from about 1:0.7 to about 400:1; and/or
d) the weight ratio of geranylgeraniol to bromelain ranges from about 1:10 to about 0.8:1 or from about 1:100 to about 8:1.

138. The composition of claim 126, wherein the composition is a toothpaste.

139. The toothpaste composition of claim 138, wherein the composition further comprises one or more selected from the group consisting of water, an abrasive, a thickening agent, a terpene essential oil, a non-caloric sweetener, a humectant, a foaming agent, and an emulsifier.

140. The toothpaste composition of claim 139, wherein:
i) the abrasive is calcium phosphate, calcium carbonate, sodium bicarbonate, hydrated silica, or any combination thereof, wherein the thickening agent is cellulose gum, xanthan gum maltodextrin, or any combination thereof;
ii) the terpene essential oil is spearmint oil, wintergreen oil or peppermint oil;
iii) the non-caloric sweetener is a sugar alcohol, sucralose, rebaudioside A, mogrosides, or any combination thereof;
iv) the humectant is glycerin, sorbitol, triacetin, or any combination thereof; and/or
v) the emulsifier is cocamidopropyl betaine, quillaja saponin, yucca saponin, sodium coco sulfate, lecithin, triacetin, or any combination thereof.

141. The toothpaste composition of claim 140, wherein:
a) the peppermint oil comprises menthol;
b) the sugar alcohol is sorbitol, erythritol, xylitol, or any combination thereof; and/or
c) the composition further comprises annatto seed extract, colloidal silver, grape seed extract, Lactobacillus paracasei, or a combination thereof.

142. The toothpaste composition of claim 138, wherein the composition comprises:
i) about 10% w/w to about 90% w/w calcium phosphate,
ii) up to about 2% w/w cellulose gum,
iii) about 0.5% to about 2% w/w peppermint oil,
iv) about 5% to about 30% w/w glycerin,
v) about 0.2% to about 2% w/w quillaja saponin or yucca saponin,
vi) about 5% to about 40% w/w xylitol,
vii) less than about 1% w/w annatto seed extract,
viii) less than about 1% w/w colloidal silver,
ix) less than about 1% w/w grape seed extract,
x) less than about 1% w/w Lactobacillus paracasei, and/or
xi) about 5% to about 30% w/w water.

143. The toothpaste composition of claim 138, wherein the composition is substantially free of sodium dodecyl sulfate (SDS) or sodium lauryl sulfate (SLS).

144. The toothpaste composition of claim 138, wherein the composition comprises:
i) about 0.5% to about 5.0% w/w geranylgeraniol,
ii) about 5% to about 40% w/w xylitol,
iii) up to about 40% w/w calcium phosphate,
iv) up to about 10% w/w glycerin,
v) about 0.5% to about 2% w/w peppermint oil,
vi) about 0.5% to about 2% w/w cellulose gum,
vii) about 0.5% to about 2% w/w quillaja saponin or yucca saponin, and
viii) about 5% to about 30% w/w water,
wherein the composition is substantially free of sodium dodecyl sulfate (SDS) or sodium lauryl sulfate (SLS).

145. The toothpaste composition of claim 144, wherein the composition comprises:
i) about 1% w/w geranylgeraniol,
ii) about 35% w/w xylitol,
iii) about 30% w/w calcium phosphate,
iv) about 5% w/w glycerin,
v) about 1% w/w peppermint oil,
vi) about 1% w/w cellulose gum,
vii) about 1% w/w quillaja saponin or yucca saponin, and
viii) about 25% w/w water,
wherein the composition is substantially free of sodium dodecyl sulfate (SDS) or sodium lauryl sulfate (SLS).

146. A disposable toothbrush, such as for use in, or maintaining, oral hygiene, comprising an abrasive cleaning surface or bristles impregnated with the composition of claim 126.

147. The disposable toothbrush of claim 146, comprising:
i) about 1.0% to about 5.0% w/w geranylgeraniol,
ii) about 30% to about 40% w/w xylitol,
iii) about 10% to about 15% w/w calcium phosphate,
iv) about 20% to about 30% w/w glycerin,
v) about 1% to about 2% w/w peppermint oils,
vi) about 1% to about 2% w/w cellulose gum,
vii) about 1% to about 2% w/w quillaja saponin or yucca saponin, and
viii) about 15% to about 25% w/w water,
wherein the composition is substantially free, or free, of sodium dodecyl sulfate (SDS) or sodium lauryl sulfate (SLS).

148. A dental device or an oral care kit comprising the composition of claim 126, wherein the dental device is a dental floss, a tongue scraper, a dental pick, a mouth guard, or an orthodontic corrective device.

149. A method of:
a. Promoting gum growth, renewal, wound healing, or tissue repair;
b. Removing gum adhesives;
c. Preventing gum decay;
d. Maintaining gum energetics;
e. Improving oral health;
f. Reducing plaque accumulation;
g. Reducing or inhibiting gingivitis;
h. Inhibiting or disrupting microbial biofilm formation in the oral cavity;
i. Reducing or inhibiting formation of dental caries;
j. Reducing levels of periodontopathogens;
k. Reducing levels of anaerobes;
l. Reducing levels of acid producing bacteria;
m. Increasing relative levels of arginolytic bacteria;
n. Immunizing the teeth against cariogenic bacteria;
o. Reducing levels of lactic acid;
p. Promoting salivary production;
q Treating, relieving, or reducing dry mouth;
r. Reducing, repairing, or inhibiting early enamel lesions;
S. Promoting dentin regeneration;
t. Reducing or inhibiting demineralization or promoting remineralization of the teeth;
u. Reducing hypersensitivity of the teeth;
v. Reducing periodontal pocket depth;
w. Reducing or treating bleeding on dental probing;
x. Reducing or inhibiting clinical attachment loss and improving clinical attachment;
y. Reducing halitosis;
z. Reducing tooth staining;
aa. Enhancing salivary CoQ₁₀;
bb. Promoting healing of sores or cuts in the mouth or promoting wound healing of gum or enamel;
cc. Cleaning the teeth and oral cavity;
dd. Promoting systemic health, including cardiovascular health;
ee. Preventing or restoring buccal root death or gum death;
ff. Reducing severity of gingival inflammation;
gg. preventing or treating a jaw-bone related syndrome or disease,
hh. promoting wound healing of gum or enamel,
ii. restoring the root canal,
jj. preventing or treating dental caries, root caries, tooth fracture, root fracture, cervical abrasion, tooth wearing, or gum or enamel loss, or
kk. preventing or treating dry socket (alveolar osteitis),
or any combination thereof, comprising applying the oral composition of claim 126, wherein to the gum, tooth, tooth tissue, enamel, oral cavity, or tooth socket.

150. A dental product comprising the composition of claim 126, wherein the dental product is a dental gel, gel sponge, a bone cement, a dental bonding agent, a dental porcelain powder, a pulp capping material, a retrograde filling material, a root canal filling material, or a composite resin filling material.

151. A method of:
a. preventing or treating a jaw-bone related syndrome or disease,
b. promoting wound healing of gum or enamel,
c. restoring the root canal,
d. preventing or treating dental caries, root caries, tooth fracture, root fracture, cervical abrasion, tooth wearing, or gum or enamel loss, or
e. preventing or treating dry socket (alveolar osteitis),
to a subject in need of, wherein the method comprises applying the dental product of claim 150 to the gum, tooth, tooth tissue, enamel, oral cavity, or tooth socket of the subject.
